(19) United States
(12) Patent Application Publication
(10) Pub. No.: US 2023/0218706 A1
(43) Pub. Date: Jul. 13, 2023

(54) LARAZOTIDE DERIVATIVES COMPRISING D-AMINO ACIDS

(71) Applicant: 9 Meters Biopharma, Inc., Raleigh, NC (US)

(72) Inventors: Jay P. MADAN, Raleigh, NC (US); Balasingham RADHAKRISHNAN, Raleigh, NC (US); Sandeep LAUMAS, Raleigh, NC (US); Christopher PRIOR, Raleigh, NC (US); Anthony BLIKSLAGER, Raleigh, NC (US); Patrick H. GRIFFIN, Raleigh, NC (US); Nir BARAK, Tel Aviv (IL); Sireesh APPAJOSYULA, Raleigh, NC (US)

(21) Appl. No.: 17/996,228

(22) PCT Filed: Apr. 15, 2021
(86) PCT No.: PCT/US2021/027410

§ 371 Date: Oct. 14, 2022

Related U.S. Application Data
(60) Provisional application No. 63/114,756, filed on Nov. 17, 2020.
(60) Provisional application No. 63/010,135, filed on Apr. 15, 2020.

(51) Int. Cl. A61K 38/08; A61K 47/38; A61P 11/00

(52) U.S. Cl. CPC A61K 38/08; A61K 47/38; A61P 11/00

(57) ABSTRACT

The present invention provides compositions comprising an effective amount of a peptide having the amino acid sequence Gly-Gly-(d)Val-(d)Leu-(d)Val-(d)Gln-(d)Pro-Gly (SEQ ID NO: 6) to promote tight junction integrity, or a pharmaceutically acceptable salt thereof, and a pharmaceutically-acceptable carrier. The present invention further provides methods of using the larazotide derivative compositions for promoting tight junction integrity in patients in need thereof.

### FIELD OF THE INVENTION

The present invention provides compositions, formulations and methods for treating and preventing conditions associated with tight junction permeability, including of the intestinal epithelium.

### CROSS-REFERENCE TO RELATED APPLICATIONS

This application claims priority to, and the benefit of, U.S. Provisional Application No. 63/010,135, filed Apr. 15, 2020 and U.S. Provisional Application No. 63/114,756, filed Nov. 17, 2020, which is hereby incorporated by reference in its entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (Filename: “NMT-024PC_ST25.txt”; Date recorded: Apr. 15, 2021; File size: 2,378 bytes).

### BACKGROUND

The intestinal epithelium is the layer of cells that forms the luminal surface of the small and large intestines of the gastrointestinal (GI) tract, and represents the largest interface (more than 400 m²) between the external environment and the internal milieu. The intestinal epithelium has two important functions: absorbing nutrients and providing a barrier against harmful environmental substances such as bacteria, viruses, toxins, and food allergens.

The barrier properties of the intestinal epithelium are regulated by specialized plasma membrane structures known as tight junctions. Alterations in tight junctions can result in disruptions of the intestinal barrier functions and increased intestinal permeability. An intact intestinal barrier prevents the permeation of pathogens, antigens, endotoxins, and other pro-inflammatory substances into the body, whereas intestinal dis-integrity allows their entry, which may trigger local or systemic inflammatory disease.

Larazotide is a peptide agent that promotes tight junction integrity. Larazotide has the amino acid sequence: Gly Val Leu Val Gln Pro Gly (SEQ ID NO: 1), and can be formulated for targeted release in affected portions of the GI (e.g., small intestine and/or large intestine) or delivered to other tissues that exhibit reduced integrity of tight junctions. Larazotide has been described as exhibiting an inverse dose response, where higher doses show an attenuated activity or no activity at all. This inverse dose response may limit the overall efficacy of the drug and requires undesirable dosing schedules.

Accordingly, there remains a need for effective treatments for epithelial or endothelial barrier dysfunction.

### SUMMARY OF ASPECTS OF THE INVENTION

The present invention provides pharmaceutical compositions and methods involving improving tight junction integrity in tissues. The pharmaceutical composition comprises an effective amount of a peptide having the amino acid sequence of SEQ ID NO: 1 (Gly-Gly-Val-Leu-Val-Gln-Pro-Gly) with one or more (d)-amino acids to promote tight junction integrity. In various embodiments, the pharmaceutical composition comprises a peptide having the amino acid sequence Gly-Gly-(d)Val-(d)Leu-(d)Val-(d)Gln-(d)Pro-Gly (SEQ ID NO: 6). This peptide, also referred to herein as “(d)-larazotide” or “all d-larazotide,” is effective for promoting tight junction integrity of epithelial and endothelial tissues at substantially lower concentrations, as compared to larazotide. Thus, in various embodiments, the pharmaceutical composition contains less than about 0.5 mg of the peptide. For example, the composition can contain about 0.25 mg or less of the peptide, or about 0.1 mg or less of the peptide. In addition, in some embodiments, (d)-larazotide demonstrates reduced inhibition by peptide fragments (e.g., produced by brush border enzymes), and thus exhibits a less prominent inverse dose response. In these embodiments, the peptide is more effective of higher concentrations than larazotide. Thus, in some embodiments, the present invention contemplates that the pharmaceutical composition contains more than about 0.5 mg of the peptide. For example, the composition can contain about 0.75 mg of the peptide or more, or about 1.0 mg of the peptide or more, or about 2.0 mg or more of the peptide.

In various embodiments, the peptide is formulated for intestinal, parenteral, intranasal or pulmonary delivery, to promote integrity of epithelial and/or endothelial tight junctions in target tissue.

In various embodiments, the composition is formulated for delivery to the intestinal tract, such as the small intestine. For example, the composition can be formulated for delivery to one or more of the duodenum, jejunum, and/or the ileum. Alternatively or in addition, the composition is formulated for delivery to the large intestine. Specifically, the composition can be formulated for delivery to one or more of the cecum, the ascending colon, the transverse colon, the descending colon, and/or the sigmoid colon.

For example, with respect to intestinal delivery, the peptide can be formulated for targeted release, or can be formulated for a sustained release or modified release. In some embodiments, the present invention contemplates a delayed-release formulation, to begin release at a target region of the gastrointestinal tract. In some embodiments, the composition contains peptide-containing beads, which may have a coating that is stable in gastric fluid and unstable in intestinal fluid so as to avoid release in the stomach, but substantially release the peptide in one or more target location(s) of the small intestine, or to initiate release of the peptide in a target region of the small intestine.

In various other embodiments, the peptide is formulated for delivery by other routes, such as pulmonary delivery, parenteral delivery, intranasal delivery, and ophthalmic delivery.

In various aspects and embodiments, the invention provides methods for treating various biological conditions involving dysfunctional epithelial and/or endothelial barriers, inflammatory conditions, and conditions impacted by the gut microbiome (e.g., intestinal dysbiosis), for example. Such conditions include, but are not limited to celiac disease, Inflammatory Bowel Disease (IBD) (e.g., Crohn's Disease or Ulcerative Colitis), environmental enteropathy, necrotizing enterocolitis, intestinal ischemia, fatty liver disease (including, but not limited to NAFLD, NASH, ASH), diabetes, insulin resistance, hypertriglyceridemia, chronic kidney disease, IgA nephropathy, inflammatory condition of the respiratory tract (e.g., asthma, COPD, pulmonary fibrosis, cystic fibrosis, ALI, ARDS, emphysema, bronchitis, pneumonia, lung cancer, or a respiratory infection), autoimmune disease (e.g., rheumatoid arthritis, juvenile rheumatoid arthritis, lupus, type 1 diabetes, or multiple sclerosis), cancer including for patients undergoing immunotherapy or chemotherapy, and conditions of the central nervous system (e.g., Parkinson's disease, Alzheimer's disease, Multiple Sclerosis, or dementia), multi-organ failure, conditions associated with receiving total parenteral nutrition, and other inflammatory diseases (e.g., Kawasaki disease, Mis-C, and systemic inflammatory response syndrome).

Other aspects and embodiments of the invention will be apparent from the following detailed description.

### BRIEF DESCRIPTION OF THE DRAWINGS

FIG. 1 depicts the results of an ex vivo assay that measured transepithelial electrical resistance (TEER) of ischemic-injured porcine jejunum, and shows that larazotide induces repair of ischemic-injured intestine in a “bell-shaped” dose-dependent manner. Accumulation of larazotide fragments at higher doses may inhibit larazotide action.

FIG. 2 shows the results of an ex vivo test model measuring the recovery of an ischemic-injured pig jejunum tissue with respect to application of several larazotide-derived analogs.

FIG. 3 depicts the results of an ex vivo test measuring the recovery effects of Analogs #5 and 6 for recovery of ischemic-injured pig jejunum tissue.

FIG. 4 shows the differences between the degradation profiles of larazotide against Analog #6 ((d)-larazotide) over time in ischemic-injured pig jejunum tissue.

FIG. 5 depicts the results of an experiment in which larazotide and Analog #6 were both applied in 1 μM concentrations to ischemic-injured intestinal tissue, and transepithelial/transendothelial electrical resistance was measured over a recovery period of 240 minutes.

FIG. 6 depicts an experiment in which transepithelial/transendothelial electrical resistance was measured when Analog #6 was applied to ischemic-injured intestinal tissue at concentrations of 0.01 μM, 0.1 μM, 1 μM, and 10 μM, respectively. Larazotide applied at a concentration of 1 μM was used as a control.

FIG. 7 depicts the inhibitory effects of larazotide fragments when larazotide is applied to ischemic-injured intestinal tissue alone and in combination with the fragments: 1 μM larazotide; 10 μM Fragment #1; 10 μM Fragment #2; 10 μM Fragment #1+1 μM larazotide; and 10 μM Fragment #2+1 μM larazotide. Non-ischemic and ischemic intestinal tissue that did not receive active agent were used as controls. Transepithelial/transendothelial electrical resistance was measured over a recovery period of 240 minutes.

FIG. 8 depicts the results of the (d)-larazotide analog applied with and without the fragment A6F1 (Fragment #1 of Analog #6) as follows: 0.1 μM Analog #6; 1 μM Analog #6; 10 μM A6F1; 10 μM A6F1+0.1 μM Analog #6; and a non-ischemic tissue control.

FIG. 9 depicts the results of the (d)-larazotide analog applied with and without the fragment A6F2 (Fragment #2 of Analog #6) as follows: 0.1 μM Analog #6; 1 μM Analog #6; 10 μM A6F2; 10 μM A6F2+0.1 μM Analog #6; and a non-ischemic tissue control.

FIG. 10 depicts the results of FIGS. 8 and 9 combined, in which both A6F1 and A6F2 fragments were applied in conjunction with Analog #6.

FIG. 11 shows the changes in body weight over a course of 21 days after BLM administration when the mice were given vehicle, larazotide, or all d-larazotide.

FIG. 12 depicts survival percentages over a course of 21 days after BLM administration when the mice were given vehicle, larazotide, or all d-larazotide.

FIG. 13 shows body weight of the pulmonary fibrosis model mice on the day of sacrifice (Day 21).

FIG. 14 depicts left lung weight of the pulmonary fibrosis model mice on the day of sacrifice (Day 21).

FIG. 15 depicts post-caval lobe weight of the pulmonary fibrosis model mice on the day of sacrifice (Day 21).

FIG. 16 shows lung hydroxyproline concentrations calculated from the hydroxyproline standard curve. Lung hydroxyproline contents are expressed as μg per left lung.

FIG. 17 depicts photographs of stained sections of right lung tissue of pulmonary fibrosis model mice that were administered vehicle, larazotide, and all d-larazotide.

FIG. 18 depicts a plot of the graded results of the stained right lung sections per the Ashcroft evaluation and grading criteria.

### DETAILED DESCRIPTION

The present invention provides larazotide derivatives and compositions thereof that are effective at substantially lower doses than larazotide, and in various embodiments, do not demonstrate a substantial inverse dose response. Accordingly, the larazotide derivatives can be effectively delivered over a large dose range and/or with more desirable dosing schedules as compared to larazotide.

“Larazotide” is an eight amino acid peptide that has the sequence GGVLVQPG (SEQ ID NO: 1), alternatively depicted using the formula G1-G2-V3-L4-V5-Q6-P7-G8 to indicate amino acid sequence numbering. Larazotide, when made as the salt with acetic acid, is larazotide acetate. Larazotide promotes tight junction integrity of epithelial and endothelial tissues, including of the intestinal epithelium, and is being evaluated as a therapy for patients with celiac disease (CeD).

In accordance with certain aspects and embodiments, the present invention provides larazotide derivatives that confer increased resistance to exopeptidase degradation, including aminopeptidase degradation. A protease or peptidase is an enzyme that catalyzes the hydrolytic degradation of peptide bonds. Peptidases can be exopeptidases or endopeptidases. An exopeptidase catalyzes the cleavage of the terminal or penultimate peptide bond. Depending on whether the amino acid is released from the amino or the carboxy terminus, an exopeptidase is further characterized as an aminopeptidase or a carboxypeptidase, respectively. An aminopeptidase, such as an enzyme found in the brush border of the small intestine, will cleave one or more amino acids from the amino terminus of the peptide. A carboxypeptidase, such as an enzyme present in brush border and in the digestive pancreatic juice, will cleave one or more amino acids from the carboxylic end of the peptide. A peptide can undergo multiple rounds of N- or C-terminal cleavage.

Larazotide has been shown in clinical trials to exhibit significant benefit at reducing Celiac disease symptoms, particularly at the lower doses (e.g., 0.5 mg dose). See US 2016/0022760, which is hereby incorporated by reference in its entirety, and in particular for the formulations and dosages outlined therein. Higher doses (e.g., 1 mg and 2 mg doses) showed an attenuation of activity, or no activity at all. In accordance with this disclosure, it is believed that an exopeptidase such an aminopeptidase located within the brush borders of the lumen surface may create larazotide-derived fragments, including fragments missing N-terminal glycine residues. For example, the fragments GVLVQPG (SEQ ID NO: 7) (referred to herein as “Fragment 1”) and VLVQPG (SEQ ID NO: 8) (referred to herein as “Fragment 2”) are inactive as tight junction regulators. Moreover, when these two fragments are mixed with full-length larazotide, activity is completely abolished. Local buildup of these inactive larazotide fragments (due to excessive doses of larazotide) may in fact compete and block function of the peptide. This would explain clinical observations that low doses of larazotide work best by avoiding the reservoir of competing inactive fragments.

The present invention provides compounds that promote tight junction integrity (e.g., epithelial or endothelial tight junction integrity), and which display a substantially less inverse dose response at higher doses, as compared to larazotide. For example, the dose response for the larazotide derivatives is less “bell-shaped” than that for larazotide, where both lower doses and higher doses are more effective as compared to larazotide. In some embodiments, administering the pharmaceutical compositions of the present invention to patients in need, avoids substantial accumulation of inactive peptide fragments. In various embodiments, the peptide derivative of larazotide contains D amino acids. In some embodiments, the peptide derivative of larazotide contains 1, 2, 3, 4, or 5 D amino acids. D-amino acids are depicted herein using “(d)” to indicate that the following amino acid is in the D conformation. In some embodiments, the larazotide derivative has the amino acid sequence of Gly-Gly-Val-Leu-Val-Gln-(d)Pro-Gly (SEQ ID NO: 9). This peptide is referred to herein as “(d)-Pro” or (d)-Pro larazotide. In other embodiments, the larazotide derivative has the amino acid sequence of Gly-Gly-(d)Val-(d)Leu-(d)Val-(d)G1n-(d)Pro-Gly (SEQ ID NO: 6). This peptide is referred to herein as “(d)-larazotide.” As demonstrated herein, (d)-larazotide is surprisingly effective at promoting tight junction integrity at substantially lower concentrations as compared to larazotide. This is a surprising observation, since typically, replacing L amino acids with D amino acids in peptide drugs will result in a loss of potency. That is, a peptide with D amino acids would be expected to bind with lower affinity to the receptor function, as compared to peptides having the natural L amino acids.

The present invention provides larazotide derivatives that are surprisingly effective at substantially lower doses as compared to larazotide. Accordingly, the pharmaceutical compositions (or salt thereof) of the present invention can contain less than about 0.5 mg of the larazotide derivative. For example, in some embodiments, the pharmaceutical composition contains about 0.4 mg of the peptide or less, or about 0.3 mg of the peptide or less, or about 0.25 mg of the peptide of less, or about 0.2 mg of the peptide or less, or about 0.15 mg of the peptide or less, or about 0.1 mg of the peptide or less, or about 50 μg of the peptide of less, or about 25 μg of the peptide or less. In some embodiments, the pharmaceutical composition contains from about 50 μg to about 400 μg of the peptide, or from about 50 μg to about 200 μg, or from about 50 μg to about 150 μg. In some embodiments, these lower doses are applied or targeted to non-injured tissue (e.g., GI epithelium) to prevent loss of tight junction integrity, or in other embodiments, are applied to injured or inflamed tissue, to promote restoration of barrier function.

In other embodiments, the present invention contemplates a pharmaceutical composition that contains more than about 0.5 mg of peptide, and substantially reduces the inverse dose or “bell shape” response observed with larazotide. For example, in some embodiments, the pharmaceutical composition contains about 0.6 mg of the peptide or more, or about 0.75 mg of the peptide or more, or about 1.0 mg of the peptide or more, or about 1.25 mg of the peptide or more, or about 1.5 mg of the peptide or more, or about 2.0 mg of the peptide or more. In some embodiments, these doses are applied or targeted to non-injured tissue (e.g., GI epithelium) to prevent loss of tight junction integrity, or in other embodiments, are applied to injured or inflamed tissue, to promote restoration of barrier function.

In some embodiments, the peptide (e.g., (d)-larazotide or (d)-Pro), is administered at about 0.5 mg. For example, the peptide can be more effective at 0.5 mg doses than larazotide.

In various embodiments, the larazotide derivatives of the present invention exhibit increased resistance to peptidase degradation as compared to larazotide (the peptide of SEQ ID NO: 1). The degree of resistance can be quantified using any suitable peptidase activity assay. One of skill in the art will appreciate the various quantitative and qualitative methods in which protein degradation may be measured in order to determine susceptibility to peptidase activity. In some embodiments, the peptide demonstrates resistance to exopeptidase, aminopeptidase or caroboxypeptidase activity, and in some embodiments, a human aminopeptidase found in the brush borders of the human intestinal lumen surface. In some embodiments, the peptide demonstrates resistance to a carboxypeptidase, such as a human carboxypeptidase. In some embodiments, the caroboxypeptidase is a proliase, a proline specific exopeptidase or endopeptidase. In some embodiments, the exopeptidase is a C-terminal proline-specific exopeptidase (e.g., carboxypeptidase P).

In various embodiments, the invention provides methods for promoting tight junction integrity of a tissue, including tight junction integrity of epithelial or endothelial cells, by administering a peptide or pharmaceutical composition described herein to a subject or patient. The terms “subject” and “patient” are used interchangeably herein, and generally refer to mammalian subjects/patients. In various embodiments the subject is a human subjects. Thus, the composition may be formulated for administration to the gastrointestinal tract (GI), parenteral delivery, intra-nasal, buccal, ophthalmic or pulmonary delivery.

In some embodiments, the peptide or pharmaceutical composition is administered to the gastrointestinal tract (GI) to prevent or reduce gastrointestinal epithelial permeability and/or to reduce microbiome dysbiosis. In some embodiments, the peptide or pharmaceutical composition is administered to prevent or reduce epithelial permeability in other tissues. Pharmaceutical compositions can be formulated for targeted release in affected portions of the GI (e.g., small intestine and/or large intestine). In other embodiments, larazotide derivatives are administered systemically (e.g., intravenously or by subcutaneous injection). In some embodiments, the peptide composition is administered to the lungs as a solution aerosol or powder. In some embodiments, the peptide composition is administered to the nasal epithelium as a nasal solution or nasal emulsion. In some embodiments, the peptide composition is administered to the oral cavity epithelium as a liquid or buccal tablet solution. In some embodiments, the peptide composition is administered to the ocular surface or intraocularly.

Larazotide derivatives of the present invention may be administered in any suitable form, including as a salt. For example, peptides may be administered as an acetate salt. Alternative salts may be employed, including any pharmaceutically acceptable salt such as those listed in Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

In various embodiments, the peptides are formulated as pharmaceutical compositions, which can take the form of tablets, pills, pellets, capsules, capsules containing liquids, capsules containing multiparticulates, powders, solutions, emulsion, drops, suppositories, emulsions, aerosols, sprays, suspensions, delayed-release formulations, sustained-release formulations, modified release formulations, controlled-release formulations, or any other form suitable for use.

In some embodiments, the pharmaceutical compositions are formulated as a composition adapted for parenteral administration. Dosage forms suitable for parenteral administration (e.g. intravenous, intramuscular, subcutaneous or intraperitoneal injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents. In these embodiments, the compositions can be effective for treating conditions involving systemic inflammation or injured or inflamed endothelial tissue (e.g., vasculitis).

In some embodiments, the compositions are administered to a subject by contacting the epithelial tissues or mucosal surfaces of the gastrointestinal tract. For example, the compositions may be formulated for delivery to one or more of the small intestine and large intestine. By targeting release of the peptide in the affected region(s) (e.g. duodenum, jejunum and ileum, colon transversum, colon descendens, colon ascendens, colon sigmoidenum and cecum), tight junction dis-integrity or microbial dysbiosis at any portion of the GI can be ameliorated. Targeted delivery of the peptide can be achieved by coating beads or particles with the peptide, along with a delayed-release coating that prevents release in the stomach and degrades at or near the targeted location(s).

In some embodiments, the peptide is formulated for sustained or modified or controlled delivery in one or more locations of the GI. For Example, the present invention contemplates a sustained or controlled release formulation that may functionally release the peptide in the small and/or large intestine over the course of at least about 2 hours, or over the course of at least about 2.5 hours, or over the course of at least about 3 hours, or over the course of at least about 4 hours, or over the course of at least about 5 hours. In some embodiments, the sustained or controlled release composition begins to release peptide starting within about 10 to about 30 minutes of exposure to simulated intestinal fluid, with release of peptide continuing for at least about 180 minutes, or at least about 210 minutes, or at least about 240 minutes, or at least about 280 minutes of exposure to simulated intestinal fluid. Release profiles can be prepared, for example, using compositions with different enteric polymer coats and/or different thicknesses of the polymer coats. In some embodiments, the invention provides a composition comprising an effective amount of the peptide, or salt thereof, contained within a biodegradable or erodible polymer matrix, which further comprises an enteric coating. Formulations employing a biodegradable or erodible matrix are described in WO 2021/034629, which is hereby incorporated by reference in its entirety. Further, the erodible polymer matrix can comprise a polysaccharide matrix. In some embodiments, the matrix comprises one or more of cellulose, chitin, chitosan, alginate, amylose, pectin, callose, laminarin, chrysolaminarin, xylan, arabinoxylan, mannan, fucoidan, galactomannan, xanthan gum, dextran, welan gum, gellan gum, diutan gum, pullulan, hyaluronic acid, and derivatives thereof. In further embodiments, the matrix comprises microcrystalline cellulose. In these embodiments, the composition leverages the low effective dose of the peptide (e.g., (d)-larazotide or (d)-Pro), while also minimizing any local accumulation of inactive fragments. Further, the formulation in these embodiments has the benefit of treating large surfaces of the GI with small doses of the peptide deposited continually during transit.

In various embodiments, the pharmaceutical composition may be formulated to have a delayed-release profile, i.e. not immediately release the active ingredient(s) upon ingestion; rather, postponement of the release of the active ingredient(s) until the peptide passes the stomach and is lower in the gastrointestinal tract; for example, for release in the small intestine (e.g., one or more of duodenum, jejunum, ileum) or the large intestine (e.g., one or more of cecum, ascending, transverse, descending or sigmoid portions of the colon). In an embodiment, the pharmaceutical composition is formulated to have a delayed-release profile as described in, for example, U.S. Pat. No. 8,168,594, the entire contents of which are hereby incorporated by reference.

For example, the peptide may be administered to at least the duodenum of the patient, as an oral dosage, delayed-release composition that contains the peptide. In such embodiments, the composition comprises a first population of beads having a coating that is stable in gastric fluid and unstable in intestinal fluid so as to degrade and substantially release the peptide in the duodenum. The composition may further comprise a second population of beads with a pH-dependent coating to affect release of the peptide in the jejunum and/or ileum of the patient. For example, the second population of beads may release the peptide about 30 minutes or about 45 minutes after the beads releasing peptide in the duodenum. The oral dosage composition can be in the form of a capsule or tablet. The pH-dependent coating in some embodiments is a 1:1 co-polymer of methacrylic acid and ethyl acrylate, wherein the thickness of the layer determines the release profile of each bead. The beads may have one or more additional coatings such as a base coat, a separating layer, and an overcoat layer. In these embodiments, the contents of the beads will be released in a more bolus manner at targeted locations, but the properties of (d)-larazotide or (d)-Pro will be more effective than larazotide with such release profiles.

In an exemplary oral dosage composition, an effective amount of the peptide (e.g., as the acetate salt) is provided in first delayed-release particles that are capable of releasing the peptide in the duodenum of a patient, and second delayed release particles that are capable of releasing the peptide in the jejunum of a patient. Each particle has a core particle, a coat comprising the peptide (e.g., (d)-larazotide or (d)-Pro) over the core particle, and a delayed-release coating (e.g., a 1:1 co-polymer of acrylate and methacrylate) outside the coat comprising the peptide. Whereas the first delayed-release particles release at least 70% of the peptide in the first delayed-release particles by about 60 minutes of exposure to simulated intestinal fluid having a pH of greater than 5; the second delayed-release particles release at least 70% of the peptide by about 30 and about 90 minutes of exposure to simulated intestinal fluid having a pH of greater than 5.

For patients that may have additional symptoms of ulcerative colitis and/or Crohn's disease, or symptoms that involve the large intestinal symptoms, beads may further be formulated for segments of the large intestine, including the colon. See U.S. Pat. No. 8,796,203, which is hereby incorporated by reference in its entirety. For example, in some embodiments the subject has or is at risk of environmental enteropathy as described in US 2019/0358288, which is hereby incorporated by reference in its entirety.

Generally, the delayed-release coating may degrade as a function of time without regard to the pH and/or presence of enzymes. Such a coating may comprise, for example, a water insoluble polymer. Its solubility is therefore independent of the pH. The term “pH independent” as used herein means that the permeability of the polymer and its ability to release pharmaceutical ingredients is not a function of pH and/or is only very slightly dependent on pH. Such coatings may be used to prepare, for example, sustained release formulations. Suitable water insoluble polymers include, but are not limited to, cellulose ethers, cellulose esters, or cellulose ether-esters, i.e., a cellulose derivative in which some of the hydroxy groups on the cellulose skeleton are substituted with alkyl groups and some are modified with alkanoyl groups. Examples include ethyl cellulose, acetyl cellulose, nitrocellulose, and the like. Other examples of insoluble polymers include, but are not limited to, lacquer, and acrylic and/or methacrylic ester polymers, polymers or copolymers of acrylate or methacrylate having a low quaternary ammonium content, or mixture thereof and the like. Other examples of insoluble polymers include EUDRAGIT RS®, EUDRAGIT RL®, and EUDRAGIT NE®. Insoluble polymers useful in the present invention include, for example, polyvinyl esters, polyvinyl acetals, polyacrylic acid esters, butadiene styrene copolymers, and the like.

Various types of enteric coatings for delayed yet substantial delivery of active agents to the GI tract are known. In some embodiments, the sustained-release composition includes an enteric agent that is substantially stable in acidic environments and substantially unstable in near neutral to alkaline environments. In an embodiment, the sustained-release coating contains an enteric agent that is substantially stable in gastric fluid. The enteric agent can be selected from, for example, solutions or dispersions of methacrylic acid copolymers, cellulose acetate phthalate, hydroxypropylmethyl cellulose phthalate, polyvinyl acetate phthalate, carboxymethylethylcellulose, and EUDRAGIT®-type polymer (poly(methacrylic acid, methylmethacrylate), hydroxypropyl methylcellulose acetate succinate, cellulose acetate trimellitate, shellac or other suitable enteric coating polymers. The EUDRAGIT®-type polymer include, for example, EUDRAGIT® FS 30D, L 30 D-55, L 100-55, L 100, L 12,5, L 12,5 P, RL 30 D, RL PO, RL 100, RL 12,5, RS 30 D, RS PO, RS 100, RS 12,5, NE 30 D, NE 40 D, NM 30 D, S 100, S 12,5, and S 12,5 P. In some embodiments, one or more of EUDRAGIT® FS 30D, L 30 D-55, L 100-55, L 100, L 12,5, L 12,5 P RL 30 D, RL PO, RL 100, RL 12,5, RS 30 D, RS PO, RS 100, RS 12,5, NE 30 D, NE 40 D, NM 30 D, S 100, S 12,5 and S 12,5 P is used. The enteric agent may be a combination of the foregoing solutions or dispersions. In some embodiments, the enteric agent is EUDRAGIT F30D, which comprises a co-polymer of methyl acrylate, methyl methacrylate, and methacrylic acid. The co-polymer has a ratio of free carbonyl groups to ester groups of about 1:10.

In some embodiments, the beads comprise an enteric coating that is substantially resistant to dissolution in simulated gastric fluid. The composition remains essentially intact, or may be essentially insoluble, in gastric fluid. The stability of a gastric-resistant coating can be pH dependent. For example, the enteric coating may prevent substantial release of the peptide in simulated gastric fluid as well as simulated intestinal fluid having a pH of about 5.5. In some embodiments, the matrix provides for the sustained release of the peptide in simulated intestinal fluid having a pH of about 6 or more, such as from about 6.5 to about 7.0. Thus, the enteric coating is stable in simulated gastric fluid but unstable in simulated intestinal fluid having a pH above about 6.0. The enteric coating in such embodiments does not substantially release peptide in the duodenum, but delays release until the composition enters the jejunum, and thereafter providing a sustained release in the jejunum and ileum.

In some embodiments, the composition is a capsule for oral delivery comprising a population of beads, the population of beads comprising an effective amount of the peptide (e.g., (d)-larazotide or (d)-Pro or salt thereof) contained within an erodible polymer matrix, the beads further comprising an enteric coating, which may comprise a co-polymer of methyl acrylate, methyl methacrylate, and methacrylic acid. The ratio of free carbonyl groups to ester groups in the co-polymer may be about 1:10 (e.g., EUDRAGIT F30D). In such embodiments, the enteric coating may be from about 20% to about 30% of the total weight of the composition. In some embodiments, the erodible matrix comprises microcrystalline cellulose. In some embodiments, the composition provides for less than about 15% release of peptide after about 2 hours in simulated gastric fluid. Further, the composition provides for less than about 25% release of peptide after about 2 hours in simulated intestinal fluid having a pH of about 5.5. In various embodiments, the composition releases at least about 40% but no more than about 80% of peptide after about 2 hours in simulated intestinal fluid having a pH of about 7.0. In various embodiments, 100% release in simulated intestinal fluid having a pH of about 7 is not reached until at least three hours, or in some embodiments, at least about 3.5 or at least about four hours.

In some embodiments, the pharmaceutical composition involves a coated tablet, or coated beads or granules, having a delayed-release profile as described in, for example, U.S. Pat. No. 8,168,594, the entire contents of which are hereby incorporated by reference. An exemplary enteric coating comprises a co-polymer of acrylate and methacrylate, which is a 1:1 co-polymer in some embodiments. Other fillers, binder, and plasticizers, including for seal coats or top coats, are described in U.S. Pat. No. 8,168,594, which is hereby incorporated by reference.

In various embodiments, compositions can include one or more separating layers. The separating layer separates the core tablet or particle from the delayed-release coating. The separating layer can be applied to the core by coating or layering procedures typically used with coating equipment such as a coating pan, coating granulator or in a fluidized bed apparatus using water and/or organic solvents for the coating process. As an alternative, the separating layer can be applied to the core material by using a powder coating technique. The materials for separating layers are pharmaceutically acceptable compounds such as, for instance, sugar, polyethylene glycol, polyvinylpyrrolidone, polyvinyl alcohol, polyvinyl acetate, hydroxypropyl cellulose, methyl-cellulose, ethylcellulose, hydroxypropyl methylcellulose, carboxymethylcellulose sodium and others, used alone or in mixtures. Additives such as plasticizers, colorants, pigments, fillers, anti-tacking and anti-static agents, such as for instance magnesium stearate, titanium dioxide, talc and other additives can also be included in the separating layer.

An enteric coating composition can be dispersed or dissolved in either water or in a suitable organic solvent and applied to the core particle by methods well known to those of ordinary skill in the art. One or more delayed-release coatings can be applied to the coated core particle.

The enteric coating or other coats can include one or more inert processing aids, including but not limited to talc, silicon dioxide, magnesium stearate and the like. The enteric coating compositions can also contain pharmaceutically acceptable plasticizers to obtain the desired mechanical properties such as flexibility and hardness. Such plasticizers include, but are not limited to, triacetin, citric acid esters, phthalic acid esters, dibutyl sebacate, cetyl alcohol, polyethylene glycols, polysorbates or other plasticizers.

For example, in some embodiments, the coated particles or tablets can be further covered with an overcoat layer. The overcoat materials are pharmaceutically acceptable compounds such as sugar, polyethylene glycol, polyvinylpyrrolidone, polyvinyl alcohol, polyvinyl acetate, hydroxypropyl cellulose, methylcellulose, ethylcellulose, hydroxypropyl methylcellulose, carboxymethylcellulose sodium and others, used alone or in mixtures. The overcoat materials can prevent potential agglomeration of particles coated with the enteric coating, protect the coating from cracking during the compaction process or enhance the tableting process.

Thus, in some embodiments, the matrix comprises one or more binders, fillers, or plasticizers. Such components include one or more of cellulose or cellulose derivative, fatty acid salt, or synthetic polymer. For example, the binder, filler, or plasticizer may comprise a synthetic polymer, and the polymer is optionally a co-polymer of vinyl pyrrolidine and vinyl acetate. Alternatively, the binder, filler, or plasticizer comprises a cellulose derivative, which optionally comprises one or more of ethyl cellulose, hydroxypropylmethyl cellulose, and carboxymethyl cellulose. In some embodiments, the binder, filler, or plasticizer includes a fatty acid salt, optionally selected from a C8 to C18 fatty acid salt, which is optionally a salt of stearic acid (e.g., magnesium stearate). In some embodiments, the enteric coating comprises a plasticizer, which is optionally triethyl citrate.

The oral dosage composition can be in the form of a capsule comprising granules or beads, or may be an enteric-coated tablet, or other form. In some embodiments, the composition comprises a population of beads or granules containing the matrix and an enteric coating, which may be contained within a capsule. For example, in some embodiments, the beads comprise an enteric coating comprising a co-polymer of methyl acrylate, methyl methacrylate, and methacrylic acid, and which may optionally have a ratio of free carbonyl groups to ester groups of about 1:10. Such an enteric coating may be from about 15% to about 40% by weight of the composition. In some embodiments, the enteric coating is from about 20% to about 30% by weight of the composition, or from about 20% to about 25% by weight of the composition.

The polymer matrix can be selected such that it degrades or erodes in a substantially pH independent manner. In other embodiments, the polymer matrix degrades or erodes in a pH dependent manner. An exemplary polymer matrix comprises a polysaccharide matrix, such as a matrix comprising one or more of cellulose, chitin, chitosan, alginate, amylose, pectin, callose, laminarin, chrysolaminarin, xylan, arabinoxylan, mannan, fucoidan, galactomannan, xanthan gum, dextran, welan gum, gellan gum, diutan gum, pullulan, hyaluronic acid, and derivatives thereof. Derivatives of cellulose, for example, include alkyl, hydroxyl, and carboxylated derivatives. In some embodiments, the matrix comprises microcrystalline cellulose. In still other embodiments, the matrix comprises various biodegradable synthetic polymer known in the art.

In some embodiments, such as for patients having non-responsive or refractory celiac disease or IBS, the patient may receive adjunct therapy, which in some embodiments is synergistic with larazotide treatment. In some embodiments, the additional therapeutic agent is an anti-inflammatory agent such as steroidal anti-inflammatory agents or nonsteroidal anti-inflammatory agents (NSAIDs). Steroids, particularly the adrenal corticosteroids and their synthetic analogues, are well known in the art. Examples of corticosteroids include, without limitation, hydroxyltriamcinolone, alpha-methyl dexamethasone, beta-methyl betamethasone, beclomethasone dipropionate, clobetasol valerate, desonide, desoxymethasone, dexamethasone, diflorasone diacetate, diflucortolone valerate, fluadrenolone, fluclorolone acetonide, flumethasone pivalate, fluosinolone acetonide, fluocinonide, flucortine butylester, fluocortolone, fluprednidene (fluprednylidene) acetate, flurandrenolone, halcinonide, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloropredni sone, clocortelone, clescinolone, dichlori sone, difluprednate, flucloronide, flunisolide, fluoromethalone, fluperolone, fluprednisolone, hydrocortisone, mepredni sone, paramethasone, prednisolone, prednisone, and budesonide. NSAIDs that may be used in the present invention, include but are not limited to, salicylic acid, acetyl salicylic acid, methyl salicylate, glycol salicylate, salicylmides, benzyl-2,5-diacetoxybenzoic acid, ibuprofen, fulindac, naproxen, ketoprofen, etofenamate, phenylbutazone, and indomethacin.

In an embodiment, the additional therapeutic agent is an immunosuppressive agent such as azathioprine, cyclosporin, infliximab, and alemtuzumab.

In some embodiments, the additional therapeutic agent is an antidiarrheal agent. Antidiarrheal agents suitable for use in the present invention include, but are not limited to, DPP-IV inhibitors, natural opioids, such as tincture of opium, paregoric, and codeine, synthetic opioids, such as diphenoxylate, difenoxin and loperamide, bismuth subsalicylate, lanreotide, vapreotide and octreotide, motiln antagonists, COX2 inhibitors like celecoxib, glutamine, thalidomide and traditional antidiarrheal remedies, such as kaolin, pectin, berberine and muscarinic agents.

In some embodiments, the additional therapeutic agent is an antibacterial agent such as an antibiotic. Antibiotics suitable for use in the present invention include, but are not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); monobactam antibiotics (aztreonam); and carbapenem antibiotics (ertapenem, doripenem, imipenem/cilastatin, and meropenem).

In some embodiments, the additional therapeutic agent is a probiotic. Probiotics suitable for use in the present invention include, but are not limited to, Saccharomyces boulardii; Lactobacillus rhamnosus GG; Lactobacillus plantarum 299v; Clostridium butyricum M588; Clostridium difficile VP20621 (non-toxigenic C. difficile strain); combination of Lactobacillus casei, Lactobacillus acidophilus (Bio-K+CL1285); combination of Lactobacillus casei, Lactobacillus bulgaricus, Streptococcus thermophilus (Actimel); combination of Lactobacillus acidophilus, Bifidobacterium bifidum (Florajen3); combination of Lactobacillus acidophilus, Lactobacillus bulgaricus delbrueckii subsp. bulgaricus, Lactobacillus bulgaricus casei, Lactobacillus bulgaricus plantarum, Bifidobacterium longum, Bifidobacterium infantis, Bifidobacterium breve, and Streptococcus salivarius subsp. thermophilus (VSL #3)).

In accordance with certain embodiments, the composition is administered one or more times daily to promote GI tight junction integrity in a subject. For example, the composition may be administered about once daily, about two times daily, or about three times daily. In various embodiments, a once daily to three times daily regimen is continued for a prolonged period. In some embodiments, the regimen is continued for at least about 1 month, at least about 2 months, at least about 4 months, at least about 6 months, or at least about 8 months. In some embodiments, treatment is continuous to delay or prevent disease progression or to reduce or ameliorate symptoms of a chronic disease.

In some embodiments, the subject has celiac disease, and the peptide is formulated for release in the small intestine, including the duodenum and jejunum (and optionally the ileum). Methods of treatment with larazotide formulations are disclosed US 2016/0022760, which is hereby incorporated by reference in its entirety.

In some embodiments, the subject has Inflammatory Bowel Disease (IBD), such as Crohn's Disease or Ulcerative Colitis (UC), where the composition is formulated for delivery of the peptide to affected portions of the GI.

In some embodiments, the subject has environmental enteropathy or necrotizing enterocolitis (see US 2019/0358288, which is hereby incorporated by reference in its entirety). In such embodiments, the composition is formulated for delivery of the peptide to afflicted portions of the GI, which can include the small intestine and/or large intestine.

In some embodiments, the subject has a fatty liver disease including, but not limited to NAFLD, NASH, alcoholic steatohepatitis (ASH), or a fatty liver disease resulting from hepatitis, obesity, diabetes, insulin resistance, hypertriglyceridemia, chronic kidney disease, IgA nephropathy (also known as Berger's disease), abetalipoproteinemia, glycogen storage disease, Weber-Christian disease, Wolmans disease, acute fatty liver of pregnancy, and lipodystrophy. In some embodiments, improvements in intestinal barrier function (e.g., small intestine) limit the amount of toxins such as LPS that enter circulation and which can ultimately exacerbate disease or promote disease progression. IgA nephropathy, also known as Berger's disease, is a kidney disease that occurs when IgA deposits build up in the kidneys, causing inflammation that damages kidney tissues. In still other embodiments, the subject has intestinal ischemia. See US 2019/0358289 and 2021/0069286, which are hereby incorporated by reference in their entireties.

In some embodiments, the subject has an inflammatory condition of the respiratory tract or of other non-gastrointestinal organ or tissue. The intestinal microbiome plays an essential role in immune system development and tissue homeostasis. The intestinal microbiome impacts the immune responses of the GI tract, and also impacts the immunity of distal organs, including the lung. Thus, in some embodiments the compositions described herein for administration to the GI are effective to improve GI epithelial tissue integrity and control microbial dysbiosis, which has positive impacts on the health of other tissues and organs including the lungs, skin, liver, kidneys, pancreas, heart, nervous system (e.g., CNS), etc. In some embodiments, the subject has a respiratory disease. In some embodiments, the subject has asthma, chronic obstructive pulmonary disease (COPD), an interstitial lung disease, pulmonary fibrosis (e.g., idiopathic pulmonary fibrosis), cystic fibrosis (CF), Acute Lung Injury (ALI), Acute Respiratory Distress Syndrome (ARDS), lung cancer, or a respiratory infection (including a viral infection such as coronavirus infection, Flu, or RSV infection).

As demonstrated herein, (d)-larazotide provided orally in a mouse model of pulmonary fibrosis, shows better therapeutic effect than larazotide. Thus, in some embodiments, the subject has or is at risk of pulmonary fibrosis. Pulmonary fibrosis is characterized by accumulation of excessive connective tissue in the lungs. Causes of pulmonary fibrosis include administration of drugs such as bleomycin and cyclophosphamide; exposure to certain environmental factors such as gases, asbestos and silica, and microbial infections. Some systemic inflammatory diseases such as rheumatoid arthritis and SLE may also predispose to pulmonary fibrosis. Symptoms of pulmonary fibrosis include dyspnea, non-productive cough, fever and damage to the lung cells. Pulmonary fibrosis can be diagnosed with the aid of chest radiography, high resolution computed tomographic scanning and the results of pulmonary function tests.

In some embodiments, the patient has or is at risk to develop an interstitial lung disease, such as an interstitial lung disease selected from sarcoidosis and idiopathic pulmonary fibrosis (IPF). In various embodiments, the patient has a chronic inflammatory lung disease or condition in addition to pulmonary fibrosis, or which may predispose to or exacerbate pulmonary fibrosis. Exemplary inflammatory diseases or conditions include cystic fibrosis, asthma, chronic obstructive pulmonary disease (COPD), emphysema, chronic bronchitis, and chronic pneumonia.

In some embodiments, a patient at risk of pulmonary fibrosis is at least 60 years of age, or at least 70 years of age. In some embodiments, a patient at risk is a tobacco smoker or a former tobacco smoker. In some embodiments, the patient is genetically predisposed to develop pulmonary fibrosis. In these or other embodiments, the patient has chronic gastrointestinal reflux.

In some embodiments, the patient at risk has a chronic or recurring respiratory infection. Exemplary microbial infections that may predispose to pulmonary fibrosis include infections of Pseudomonas aeruginosa, Streptococcus pneumonia, and mycobacterium (tuberculosis or non-tuberculous mycobacterium). Other microbial infections include viral infections such as parainfluenza and coronavirus, such as Sars-CoV2.

In some embodiments, the patient has sarcoidosis, which is a disease characterized by granulomas, commonly in the lungs and lymph nodes. Sarcoidosis can affect other organs. Alternatively, the patient may have IPF. IPF is a condition in which the lungs become scarred without a clear etiology.

In still other embodiments, the patient has or is at risk of drug-induced pulmonary fibrosis. For example, the patient may be undergoing treatment with an antibiotic that induces pulmonary fibrosis (e.g., nitrofurantoin), methotrexate, amiodarone, and cancer chemotherapy (e.g., bleomycin or alkylating agent such as cyclophosphamide). Thus, in some embodiments, the patient is undergoing therapy with an agent that induces pulmonary fibrosis, and treatment with the peptide is provided prophylactically.

In still other embodiments, the patient having or at risk of pulmonary fibrosis has significant occupational exposure to asbestos.

In various embodiments, the patient having pulmonary fibrosis has mild or moderate pulmonary fibrosis. In still other embodiments, the pulmonary fibrosis is severe. The severity of the disease can be determined using, for example, lung function tests (e.g., forced vital capacity, FVC), high resolution CT scan (HRCT), and/or severity of symptoms such as breathlessness and cough.

In still other embodiments, the patient has or is at risk of a respiratory condition (including those described above), and the composition comprising the peptide is formulated for administration directly to the lungs (pulmonary delivery) or for systemic delivery (e.g., parenteral delivery). In some embodiments, the patient has or at risk of Acute Lung Injury (ALI), Acute Respiratory Distress Syndrome (ARDS), interstitial lung disease (e.g., pulmonary fibrosis, such as idiopathic pulmonary fibrosis), cystic fibrosis, or a bacterial or viral infection (e.g., coronavirus infection such as SARS-CoV, influenza virus infection, or RSV infection). Exemplary formulations for pulmonary delivery, including powder and solution aerosols, are described in U.S. Pat. No. 10,723,763, which is hereby incorporated by reference in its entirety.

In some embodiments, the subject has an autoimmune disease, such as, but not limited to rheumatoid arthritis, juvenile rheumatoid arthritis, lupus, type 1 diabetes, or multiple sclerosis. Rheumatoid arthritis is a chronic inflammatory disorder in which the body's immune system attacks joint tissue and, in severe cases, internal organs. Over long periods of time, the inflammation associated with rheumatoid arthritis can cause bone erosion and joint deformity. Juvenile rheumatoid arthritis, also known as juvenile idiopathic arthritis is a type of arthritis that causes joint inflammation and stiffness for more than six weeks in a child aged 16 or younger. Lupus is a disease that occurs when the body's immune system attacks its own tissues and organs, including joints, skin, kidneys, blood cells, brain, heart and lungs. Type 1 diabetes is an autoimmune disease in which the body's immune systems attacks and destroys pancreatic cells that produce insulin. Multiple sclerosis is an autoimmune disorder in which the body's immune system attacks and destroys the protective covering of nerves. In these embodiments, the composition can be formulated for delivery to the GI as described herein, but is optionally delivered systemically (e.g., parenterally) or locally to affected tissues (including the lungs, skin, or eyes).

In some embodiments, the invention provides a method for treating or preventing neoplasms (e.g., a solid tumor or blood cancer). For example, endogenous and exogenous microorganisms can facilitate the pathogenesis of various neoplasms. See Hamada T, et al., Integration of microbiology, molecular pathology, and epidemiology: a new paradigm to explore the pathogenesis of microbiome-driven neoplasms. J Pathol 2019; 247: 615-628. In various embodiments, the cancer is a primary cancer. A primary cancer refers to cancer cells at an originating site that become clinically detectable, and may be a primary tumor. For example, the cancer may be Stage I or Stage II cancer. “Metastasis” refers to the spread of cancer from a primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. In these embodiments, the composition can be formulated for delivery to the GI as described herein, but is optionally delivered systemically (e.g., parenterally).

In some embodiments, the cancer may have an origin from any tissue. In some embodiments, the cancer may originate from skin, colon, breast, or prostate, and thus may be made up of cells that were originally skin, colon, breast, or prostate, respectively. In some embodiments, the cancer is a solid tumor such as a sarcoma or carcinoma. In some embodiments, the cancer may also be a hematological malignancy, which may be lymphoma or leukemia. In some embodiments, the primary or metastatic cancer is lung cancer, breast cancer, kidney cancer, liver cancer, prostate cancer, cervical cancer, colorectal cancer, pancreatic cancer, melanoma, ovarian cancer, bone cancer, urothelial cancer, gastric cancer, head and neck cancer, glioblastoma, head and neck squamous cell carcinoma (HNSCC), non-small cell lung carcinoma (NSCLC), small cell lung cancer (SCLC), bladder cancer, prostate cancer (e.g. hormone-refractory). In some embodiments, the cancer is melanoma, colorectal cancer, or head and neck cancer. In some embodiments, the subject has or is at risk of colorectal cancer, including at risk of recurrent colorectal cancer.

In some embodiments, the cancer is progressive, locally advanced, or metastatic carcinoma. In some embodiments, the cancer is metastatic melanoma, and may be recurrent. In some embodiments, the metastatic melanoma is stage III or IV, and may be stage IVA, IVB, or IVC. The metastasis may be regional or distant. In some embodiments, the solid tumor is a relapsed or refractory solid tumor.

In some embodiments, the subject has a neoplasm (e.g., a solid tumor or blood cancer), and may be undergoing cancer therapy, such as but not limited to chemotherapy, radiation therapy, or immunotherapy (e.g., adoptive cell therapy, CAR-T therapy, immune checkpoint inhibitor therapy). In such embodiments, the composition comprising the peptide (e.g., (d)-larazotide) can potentiate the immunotherapy, providing synergistic effects. See WO 2019/183036, which is hereby incorporated by reference in its entirety. In some embodiments, the subject is receiving, has received, or will receive therapy with an immune checkpoint inhibitor. Examples of checkpoint inhibitor drugs include, but are not limited to, ipilimumab pembrolizumab, nivolumab, cemiplimab, atezolizumab, avelumab, and durvalumab. In these embodiments, the composition can be formulated for delivery to the GI as described herein, but is optionally delivered systemically (e.g., parenterally). Preferably, the composition is delivered to the GI.

In various embodiments, administration of the composition increases or restores the efficacy of immune checkpoint inhibitor therapy. For example, in some embodiments, the subject having cancer was previously unresponsive to, or had become resistant to, an immune checkpoint inhibitor. In some embodiments, the cancer is refractory or insufficiently responsive to an immunotherapy, such as anti-CTLA-4, anti-PD-1, or anti-PD-L1 and/or PD-L2 agent. In some embodiments, the cancer subject has progressed after or during treatment with an anti-CTLA-4, anti-PD-1, or anti-PD-L1 and/or PD-L2 agent, including for example, one or more of ipilimumab, tremelimumab, pembrolizumab and nivolumab, or shown no response to such treatment for at least about 4 weeks, or at least about 8 weeks, or at least about 12 weeks of treatment.

In some embodiments, the subject is undergoing chemotherapy, and is at risk or experiencing chemotherapy-induced colitis. In various embodiments, chemotherapy drugs (in particular, high-dose chemotherapy) used to treat cancer disrupt the normal balance of bacteria in the colon, thus resulting in colitis.

In some embodiments, the subject has a condition affecting the central nervous system (CNS), including, but not limited to, neurodegenerative diseases and demyelinating diseases. In some embodiments, the disease is Parkinson's disease, Alzheimer's disease, Multiple Sclerosis, or dementia. In these embodiments, the composition can be formulated for delivery to the GI as described herein, but is optionally delivered systemically (e.g., parenterally). Preferably, the composition is delivered to the GI.

In some embodiments, the present invention contemplates treatment of subjects that are critically ill, where the critical illness is a life-threatening multisystem process with a substantial risk of mortality. In various embodiments, the subject is at risk of multi-organ failure.

In some embodiments, the subject is undergoing parenteral nutrition. In these embodiments, the composition prevents or ameliorates or treats total parenteral nutrition (TPN)-induced disorders, including, but not limited to, total parenteral nutrition (TPN)-induced intestinal mucosal atrophy or total parenteral nutrition (TPN)-induced liver disease. Total parenteral nutrition (TPN) leads to small intestinal atrophy and diminished intestinal function. Furthermore, subjects having TPN-induced liver disease can exhibit cholestasis, extramedullary hematopoiesis, and microvesicular and macrovesicular steatosis. In these embodiments, the composition can be formulated for delivery to the GI as described herein.

In still other embodiments, the subject has an inflammatory disease, such as, but not limited to, Kawasaki disease, Multisystem inflammatory syndrome in children (Mis-C), or systemic inflammatory response syndrome. Kawasaki disease, also known as mucocutaneous lymph node syndrome, is a condition that causes inflammation in the blood vessels, particularly the coronary arteries, and is most common in infants and young children. Multisystem inflammatory syndrome in children (Mis-C) is a condition of unknown cause, where different body parts can become inflamed, including the heart, lungs, kidneys, brain, skin, eyes, or gastrointestinal organs. In some embodiments, the subject has systemic inflammatory response syndrome, which can be an exaggerated defense response of the body to a noxious stressor (e.g., infection, trauma, surgery, acute inflammation, ischemia or reperfusion, or malignancy). In these embodiments, the composition can be formulated for delivery to the GI as described herein, but is optionally delivered systemically (e.g., parenterally) or locally to affected tissues (including the lungs, skin, or eyes).

In various embodiments, the composition comprising the peptide can be administered one or more times per day, such as from one to three times per day. While larazotide has generally been dosed at three times daily during clinical trials for CeD, in view of the higher stability and improved potency, the peptides described herein comprising one or more D-amino acids (e.g., (d)-larazotide or (d)-Pro larazotide) can be provided as a longer-acting analog with more convenient dosing schedule. For example, in some embodiments, the (d)-larazotide or (d)-Pro larazotide composition is administered once or twice daily, or once or twice weekly to the GI tract (e.g., orally). In some embodiments, the (d)-larazotide or (d)-Pro larazotide is administered once daily to the GI tract (e.g., orally). In some embodiments, the (d)-larazotide or (d)-Pro larazotide composition is administered about once or twice daily, or once or twice weekly, via another route, such as parenteral, intranasal, pulmonary, or ophthalmic administration.

Embodiments of the invention will now be described with reference to the following examples.

As used herein, the term “about”, unless the context requires otherwise, means±10% of an associated numerical value.

### EXAMPLES

### Example 1: Development of Larazotide Analogs with Improved Activity Profiles

### Model of Dose-Dependent Response of Larazotide

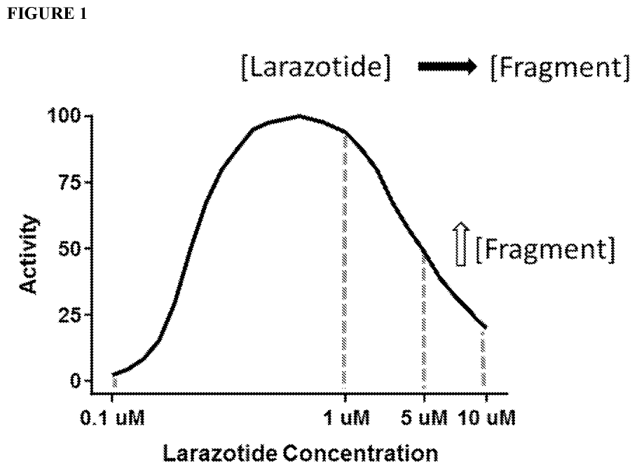

In order to evaluate the activity profile of larazotide and larozotide analogs, an ex vivo assay was employed measuring transepithelial electrical resistance (TEER) of ischemic-injured porcine jejunum. FIG. 1 shows that larazotide induces repair of ischemic-injured intestine in a “bell-shaped” dose-dependent manner. The reparative action of larazotide is attributable to tight junction reassembly. That is, the maximum and profound effect was found at the middle dose range, and there was no significant effect at the low and high dose levels. The low and high ends of the dose range show little to no effect likely due to the activity of the brush border enzyme aminopeptidase present in the mucin layer. As a result, the action of larazotide, when given as a high dose, is likely blocked by an excess concentration of inhibitory fragments. When given at a low dose, the activity of larazotide is inhibited by excessive degradation itself. This “bell-shaped” dose-activity relationship makes dosing of larazotide in the clinical setting challenging. Larazotide analogs were designed to investigate whether analogs might have more desirable dose-activity profiles.

### Larazotide Analog Design

D-larazotide analogs, chirally-modified larazotide analogs, were designed to hinder brush border enzyme recognition for degradation and prevent its fragmentation while maintaining recovery efficacy of larazotide. The following analogs were designed for ex vivo testing:

TABLE 1
List of Designed Larazotide Analogs

| Name | Single Letter Nomenclature | SEQ ID NO: |
|---|---|---|
| Larazotide | H2N-GGVLVQPG-OH | 1 |
| Analog #1 | H2N-GGV(dL)VQPG-OH | 2 |
| Analog #2 | H2N-(dV)GGVLVQPG-OH | 3 |
| Analog #3 | H2N-VGGVLVQPG-OH | 4 |
| Analog #5 | H2N-GG(dV)LVQPG-OH | 5 |
| Analog #6 | H2N-GG(dV)(dL)(dV)(dQ)(dP)G-OH | 6 |
| ((d)-larazotide) | | |

### Ex Vivo Testing of Larazotide Analogs

Several larazotide analogs were initially tested in an ex vivo model, measuring the recovery of an ischemic-injured pig jejunum tissue.

Six-to-eight-week-old Yorkshire crossbred pigs of either sex were individually housed and maintained on a commercially pelleted feed. Pigs were held off feed for 12 hours prior to experimental surgery. General anesthesia was induced with xylazine (1.5 mg/kg IM), ketamine (11 mg/kg IM), followed by mask delivery of isoflurane vaporized in 100% 02. Pigs were subsequently orotracheally intubated, followed by continued delivery of isoflurane in 100% 02 to maintain a surgical plane of anesthesia. An intravenous catheter was placed in an ear vein for delivery of fluids during surgery (lactated Ringer solution, 15 m1·kg⁻¹h⁻¹, IV). The distal jejunum was approached via a ventral midline incision. Jejunal segments were delineated by ligating the intestine at 8-10 cm intervals with 2-0 silk, and were subjected to segmental ischemia by ligating the local mesenteric blood supply with 2-0 silk for 45-minutes.

### Ussing Chamber Studies

After 45-minutes of ischemia, the pigs were euthanized, and intestinal segments were collected. Non-ischemic control tissue was also collected at this time. Following tissue collection, the seromuscular layer was stripped from the mucosa while the tissue was bathed in oxygenated (95% 02, 5% CO₂) Ringer's solution (in mmol/L: 154 Na⁺, 6.3 K⁺, 137 Cl⁻, and 24 HCO₃_{^{−}}; pH 7.4). The mucosa was subsequently mounted in 1.1-cm² aperture Ussing chambers, as described in previous studies, which are herein incorporated by reference in their entireties, Moeser, A. J.; Nighot, P. K.; Ryan, K. A.; Wooten, J. G.; Blikslager, A. T. Prostaglandin-mediated inhibition of Na+/H+ exchanger isoform 2 stimulates recovery of barrier function in ischemia-injured intestine. Am J Physiol Gastrointest Liver Physiol (2006), 291, G885-94; and Jin, Y.; Blikslager, A. T. Myosin light chain kinase mediates intestinal barrier dysfunction via occludin endocytosis during anoxia/reoxygenation injury. Am J Physiol Cell Physiol (2016), 311, C996-C1004. Tissues were bathed on both the mucosal and serosal sides with 10 mL Ringer's solution. The mucosal bathing solution contained 10 mM mannitol while the serosal side was osmotically balanced with 10 mM glucose. Bathing solutions were oxygenated (95% O₂, 5% CO₂) and circulated in water-jacketed reservoirs. The spontaneous potential difference (PD) was measured using Ringer-agar bridges connected to calomel electrodes, and the PD was short-circuited through Ag—AgCl electrodes using a voltage clamp that was corrected for fluid resistance. TER (Ω·cm²) was calculated from the spontaneous PD and short-circuit current (I_{sc}). If the spontaneous PD was between −1.0 and 1.0 mV, tissues were current clamped at ±100 μA for 5-seconds, and the PD was recorded. I_{sc} and PD were recorded at 15-minute intervals over a 4-hour experiment. After tissues were mounted on Ussing chambers, all tissues were allowed to acclimate for a period of 30-minutes to establish stable baseline measurements, after which experimental treatments of larazotide or larazotide analogs was added to the apical chamber. Tissues were monitored by measuring transepithelial resistance (TER) for 240 min. Samples of Ringers solution were collected at select timepoints and quenched with 5% trifluoroacetic acid (TFA) solution (in 80% acetonitrile (ACN): 20% DI water). Quenched samples were centrifuged at 13000×g for 5-minutes and the resulting supernatants were stored at −80° C. for future MS analysis.

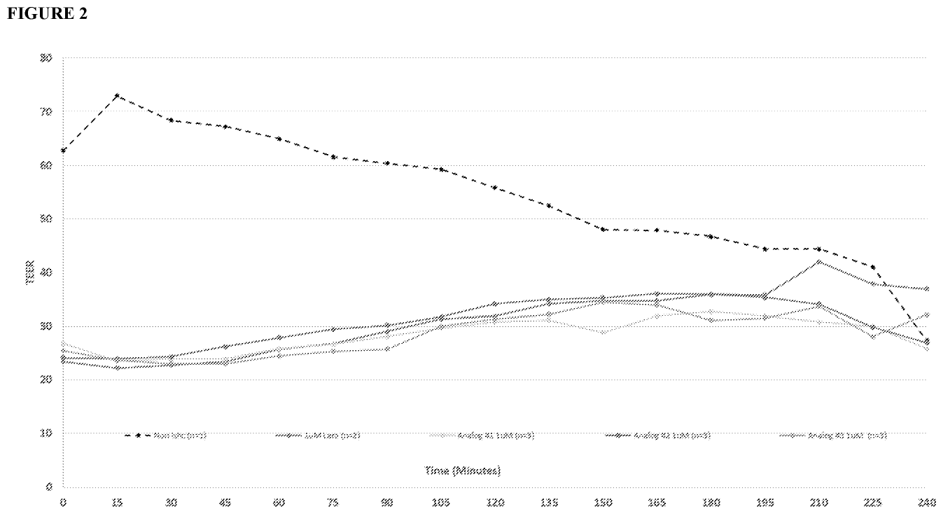

FIG. 2 depicts the results of a test of Analogs #1, 2, and 3, which showed no significant induction of recovery of ischemic-injured tissue.

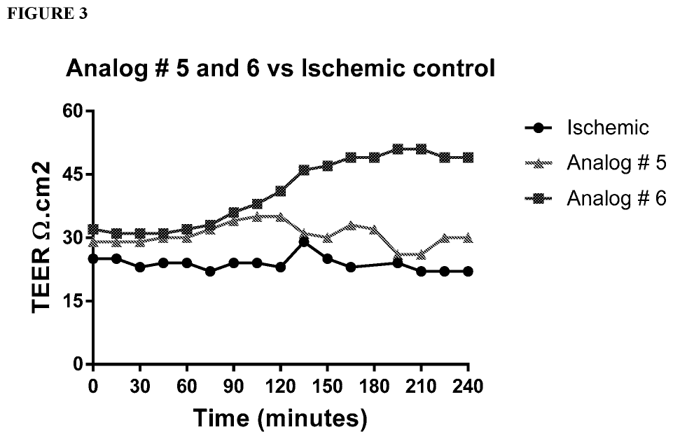

The recovery effects of Analogs #5 and 6 were also compared in an ex vivo test for recovery of ischemic-injured pig jejunum tissue. As shown in FIG. 3, Analog #6 (the all D-amino acid larazotide analog) showed a pronounced recovery effect as compared to Analog #5, which changed Val to a D-amino acid. Analogs #5 and 6 were further compared for peptide stability—Analog #5 (D-Val), Analog #6 (All D) and larazotide concentrations were measured at TO, 30, and 120 minutes after 1 μm addition of each to pig ischemic jejunum tissue. As shown in Table 2 below, the All D larazotide (Analog #6) showed significant resistance to brush border enzyme degradation as compared to larazotide and Analog #5.

TABLE 2
Ex vivo Assessment of Peptide Stability

| Time, mins | | Analog #6 | Analog #5 |
|---|---|---|---|
| (after addition) | Larazotide | (D-All) | (D-Val) |
| T = 0 | 0.508 ug/mL | 0.517 ug/ml | 0.510 ug/mL |
| T = 30 | 0.212 ug/mL | 0.436 ug/mL | 0.012 ug/mL |
| T = 120 | 0.007 ug/mL | 0.086 ug/mL | 0.005 ug/mL |

The results of the initial larazotide analog testing produced several findings. First, it was shown that extending the N-terminus of larazotide did not yield resistance to degradation by jejunum brush border enzymes. With regards to changing the chiral configuration of all (non-glycine) amino acids of larazotide to D-amino acids, this “all-D amino acid” peptide exhibited (1) a significant resistance effect to epithelial brush border enzyme degradation, and (2) a pronounced tight junction recovery effect.

### Example 2: All-D Amino Acid Larazotide Analog is More Effective than Larazotide

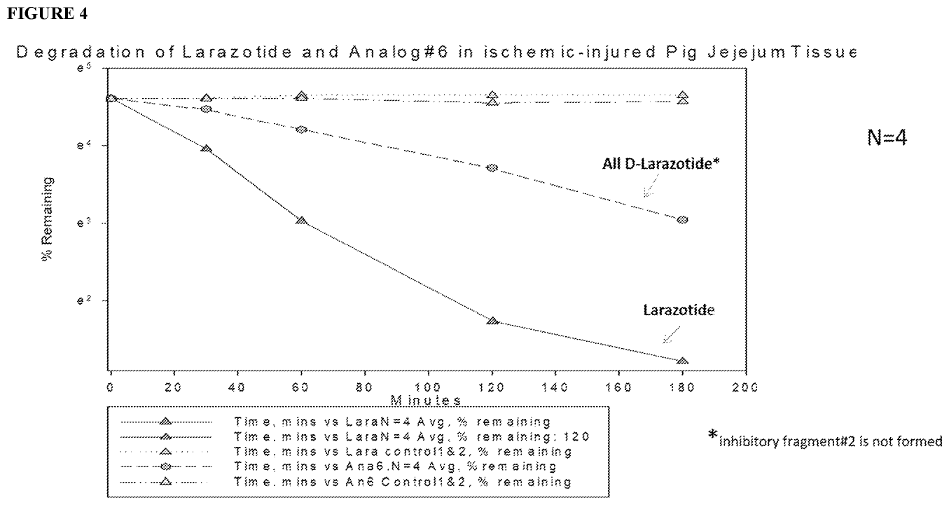

A set of experiments was conducted in order to compare the all-D amino acid larazotide analog (Analog #6) with larazotide in terms of degradation profiles, recovery effects, and dose response profiles. In an experiment evaluating the differences between the degradation profiles of larazotide against Analog #6 over time, FIG. 4 shows that Analog #6 is significantly more resistant to degradation than larazotide. That is, Analog #6 degrades significantly slower than larazotide by jejunum epithelial brush border enzymes.

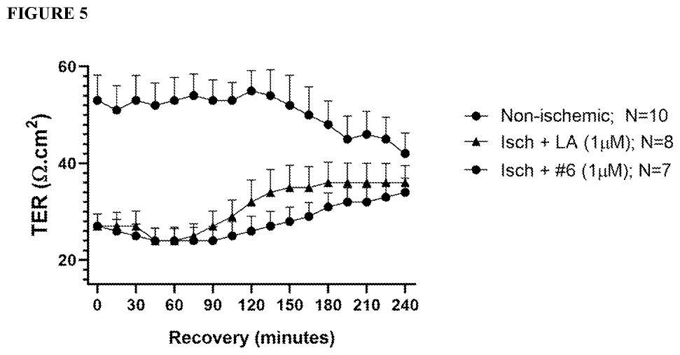

An ex vivo study evaluating whether Analog #6 can induce recovery of ischemic-injured intestine was performed, and FIG. 5 shows that the all D-amino acid larazotide analog induces recovery to substantially the same extent as larazotide over a recovery period of 240 minutes. Specifically, larazotide and Analog #6 were both applied in 1 μM concentrations to ischemic-injured intestinal tissue, and transepithelial/transendothelial electrical resistance was measured over a recovery period of 240 minutes. A non-ischemic tissue control was also conducted in which no active agent was administered. Surprisingly, the all D-amino acid larazotide analog is as effective as larazotide with regards to inducing recovery of ischemic-injured intestine. While it would be anticipated that a peptide drug having L amino acids substituted with D amino acids would lose potency due to loss of affinity to a receptor binding site, d-larazotide did not demonstrate this behavior.

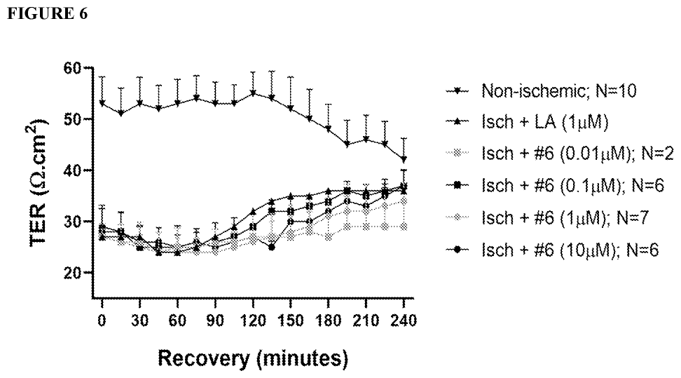

A dose response study was conducted to compare the activity profiles of Analog #6 and larazotide. Specifically, FIG. 6 depicts an experiment in which transepithelial/transendothelial electrical resistance was measured when Analog #6 was applied to ischemic-injured intestinal tissue at concentrations of 0.01 μM, 0.1 μM, 1 μM, and 10 μM, respectively. Larazotide applied at a concentration of 1 μM was used as a control. Non-ischemic tissue that did not receive any active agent was also used as a control. The results of the experiment show that Analog #6 induces the repair of injured intestine in a dose response manner different from larazotide, and did not exhibit the distinct bell-shaped dose-response of larazotide. As shown in FIG. 6, 0.1 μM of Analog #6 induced good recovery of TER, with similar activity observed with higher concentrations.

### Example 3: All-D Amino Acid Larazotide Analog-Induced Recovery is Uninhibited by Excess of D-Larazotide Fragments

Several experiments were conducted in order to determine whether Analog #6 activity would be inhibited by an excess of Analog #6 fragments, such as is the case with larazotide action.

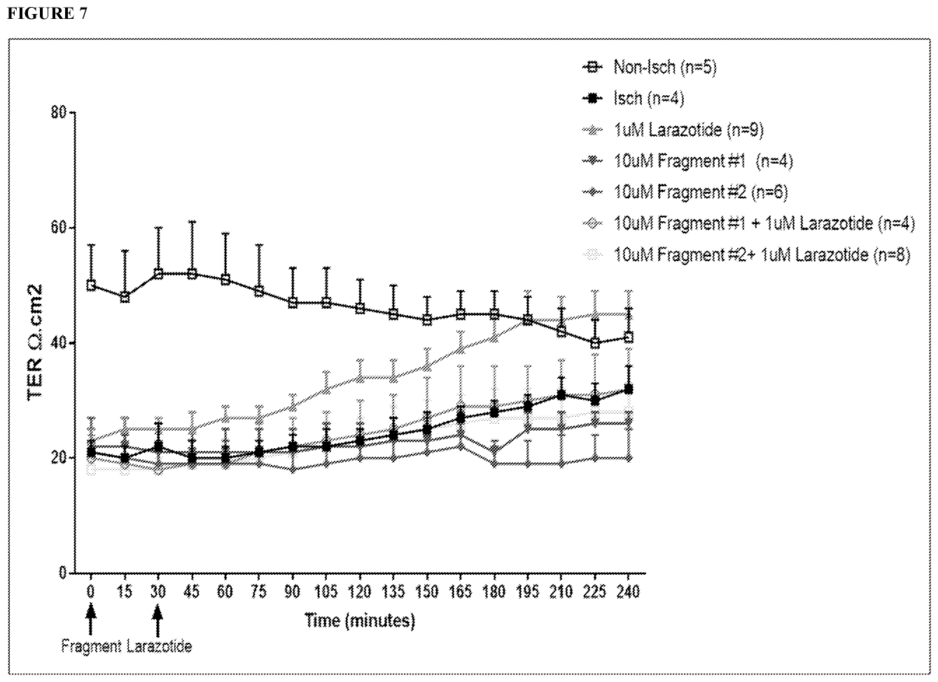

Known larazotide fragments that inhibit larazotide activity are Gly-Val-Leu-Val-Gln-Pro-Gly (Fragment 1) and Val-Leu-Val-Gln-Pro-Gly (Fragment 2). For illustrative purposes, FIG. 7 depicts the inhibitory effects of larazotide fragments (10-fold excess) when larazotide is applied to ischemic-injured intestinal tissue alone or shortly after (e.g., about 30 minutes after) addition of the fragments: 1 μM larazotide; 10 μM Fragment #1; 10 μM Fragment #2; 10 μM Fragment #1+1 μM larazotide; and 10 μM Fragment #2+1 μM larazotide. Non-ischemic and ischemic intestinal tissue that did not receive active agent were used as controls. Transepithelial/transendothelial electrical resistance was measured over a recovery period of 240 minutes.

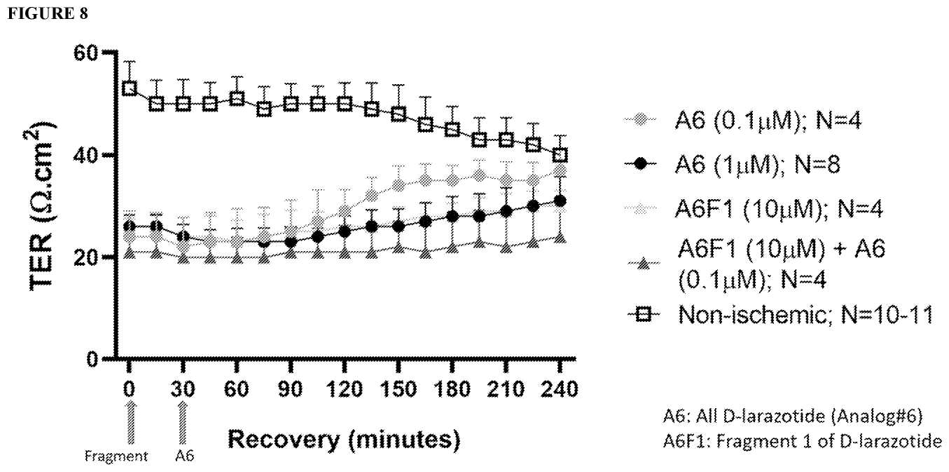

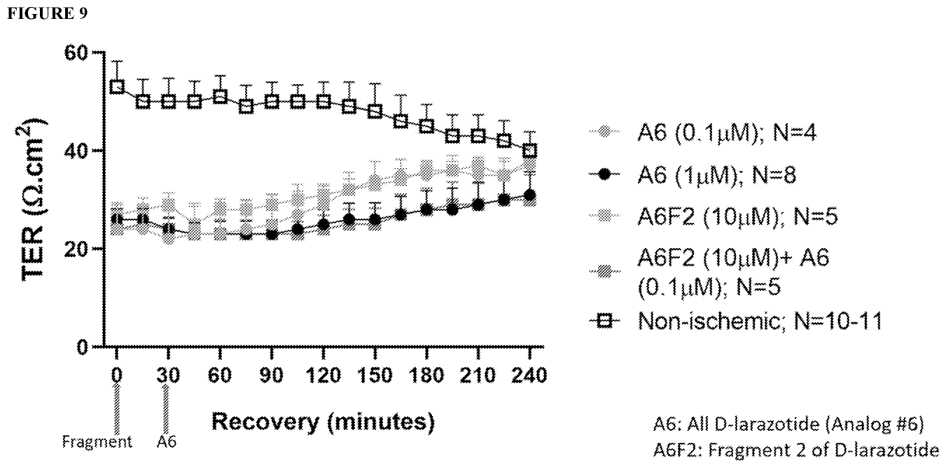

An experiment was then conducted to evaluate the effects of d-larazotide fragments on the activity of Analog #6. Specifically, the d-larazotide fragments Gly-(d)Val-(d)Leu-(d)Val-(d)Gln-(d)Pro-Gly (A6F1, Fragment #1 of Analog #6) and (d)Val-(d)Leu-(d)Val-(d)Gln-(d)Pro-Gly (A6F2, Fragment #2 of Analog #6) were applied shortly before (30 mins before) the all-D amino acid larazotide analog (Analog #6). FIG. 8 depicts the results of the all-D amino acid larazotide analog applied with and without the fragment A6F1 as follows: 0.1 μM Analog #6; 1 μM Analog #6; 10 μM A6F1; 10 μM A6F1+0.1 μM Analog #6; and a non-ischemic tissue control. The results show that 100 fold excess of A6F1 appears to have less significant impact on the action of the all-D amino acid larazotide analog (Analog #6). FIG. 9 depicts the results of the all-D amino acid larazotide analog applied with and without the fragment A6F2 as follows: 0.1 μM Analog #6; 1 μM Analog #6; 10 μM A6F2; 10 μM A6F2+0.1 μM Analog #6; and a non-ischemic tissue control. The results show that action of the all-D amino acid larazotide analog (Analog #6) does not appear to be meaningfully blocked by 100 fold excess concentration of A6F2.

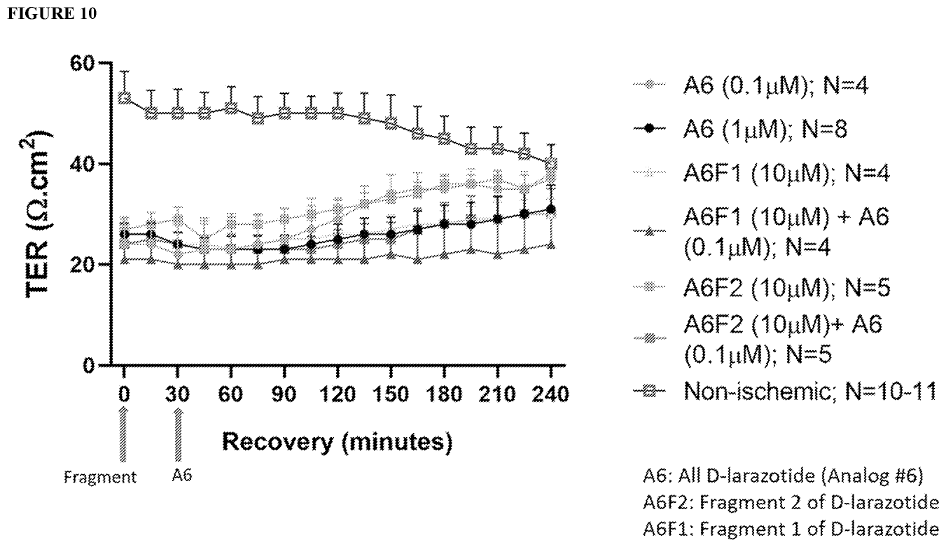

FIG. 10 depicts the results of FIGS. 8 and 9 combined, in which both A6F1 and A6F2 fragments were applied in conjunction with Analog #6. The results show that a 100-fold excess of all D-amino acid larazotide analog fragments exhibit less significant competitive inhibitory trends against the activity of Analog #6.

Overall, the results demonstrate that, as opposed to larazotide degradation fragments, all D-amino acid larazotide fragments show less competitive inhibition. Furthermore, the all D-amino acid larazotide analog facilitates tight junction recovery in injured epithelium with greater stability than larazotide and with the potential for prolonged activity as a result of reduced inhibitory fragments.

### Example 4: All D-Amino Acid Larazotide Analog Induces Recovery in Pulmonary Fibrosis Model

An experiment was conducted to evaluate the effect of larazotide and all d-amino acid larazotide on lung fibrosis in bleomycin (BLM)-induced pulmonary fibrosis model.

The experiment consisted of 3 study groups: (1) Group 1, which represented the vehicle group, consisted of 12 BLM-induced pulmonary fibrosis model mice that were orally administered vehicle (pure water) in a volume of 10 mL/kg once daily from Day 0 to 20; (2) Group 2, which represented the larazotide group, consisted of 12 BLM-induced pulmonary fibrosis model mice that were orally administered vehicle supplemented with larazotide at a dose of 1 mg/kg in a volume of 10 mL/kg once daily from Day 0 to 20; and Group 3, which represented the all d-larazotide group, consisted of 12 BLM-induced pulmonary fibrosis model mice that were orally administered vehicle supplemented with all d-larazotide at a dose of 1 mg/kg in a volume of 10 mL/kg once daily from Day 0 to 20.

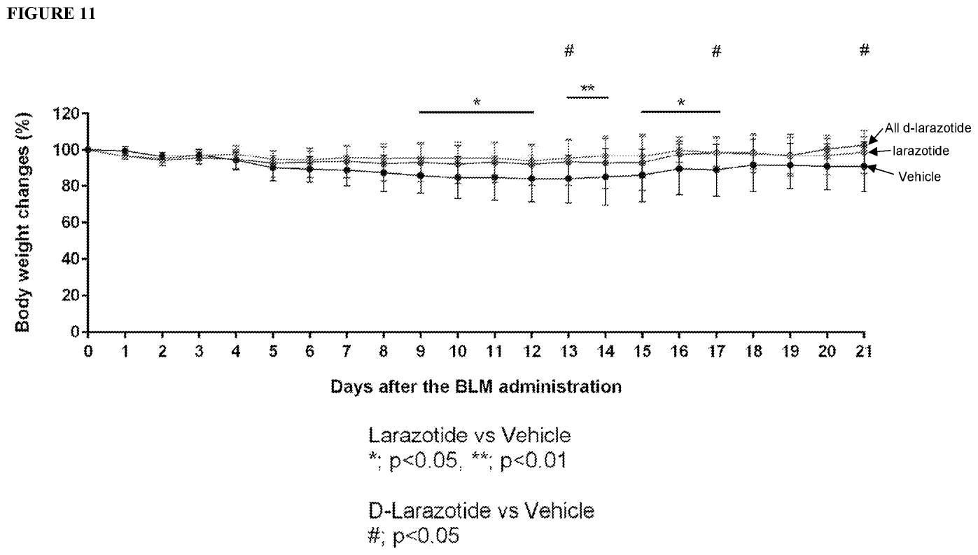

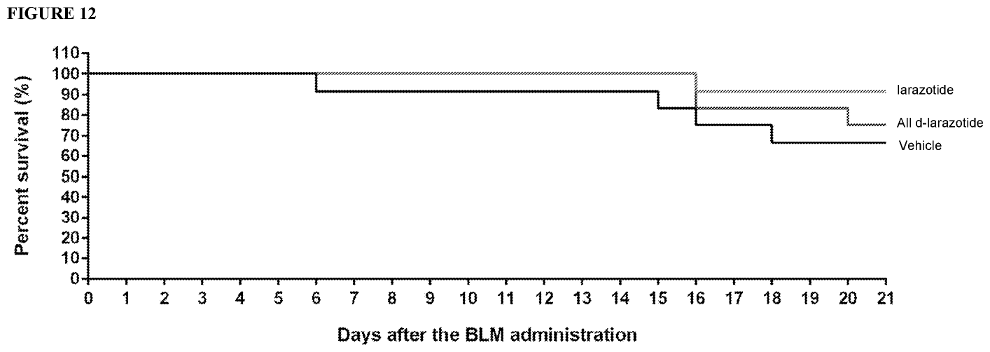

The animals were monitored daily for viability, clinical signs, and behavior. Body weight was recorded daily, and mice were observed for significant clinical signs of toxicity, moribundity, and mortality. FIG. 11 depicts the changes in body weight over the course of the study in days after BLM administration when the mice were given vehicle, larazotide, or all d-larazotide. FIG. 12 shows survival over the course of the study.

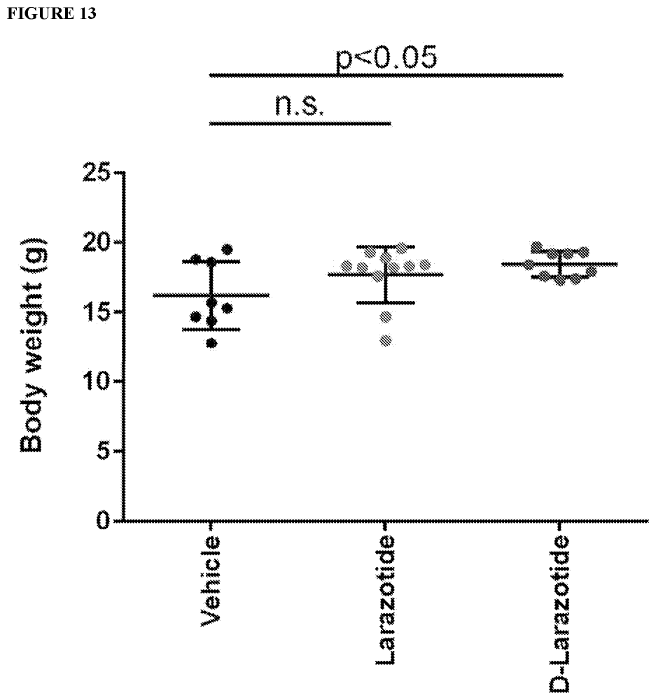

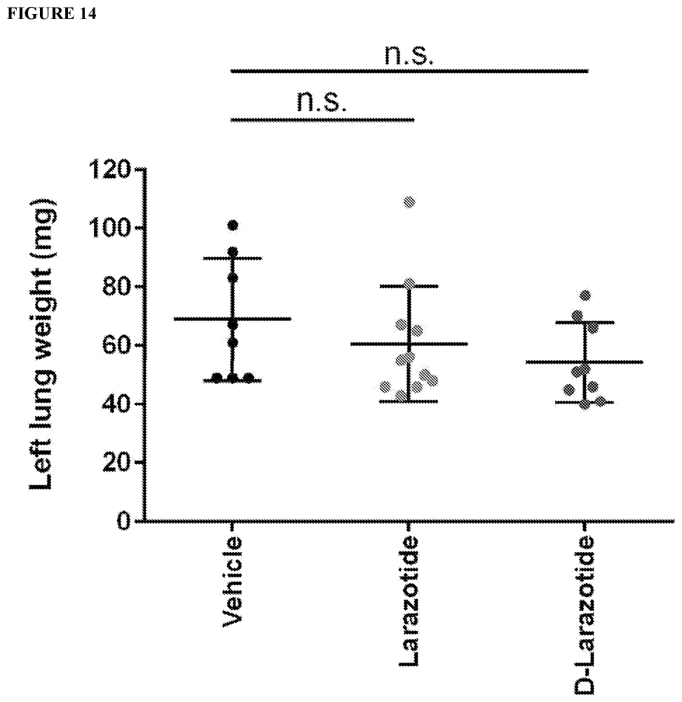

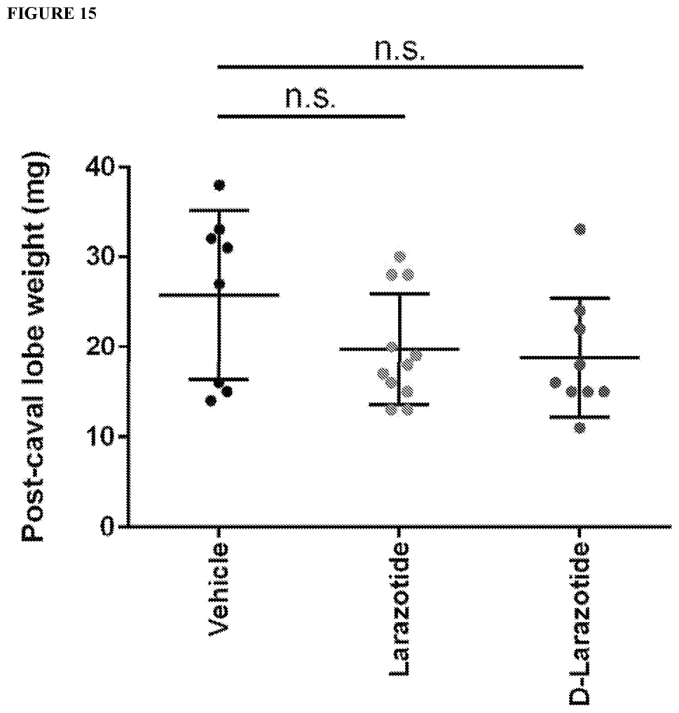

The animals were sacrificed on Day 21 (FIG. 13 shows body weight of the mice on the day of sacrifice), and lung samples were collected. Specifically, left and post-caval lobe bronchus were ligated to avoid leakage of the fixative. FIG. 14 shows left lung weight, and FIG. 15 shows post-caval lobe weight of the mice on the day of sacrifice. An indwelling needle was inserted into the trachea and connected to instillation route of syringe. The syringe was then loaded 10% neutral buffered formalin and kept at the height of 20 cm. Then, superior (A), middle (B) and inferior lobes (C) were instilled 10% neutral buffered formalin and ligated after inflation. Three fixed lobes (for histological analyses), unfixed left lung (E) (for biochemistry) and unfixed post-caval lobe (D) were harvested. Two unfixed lobes were washed with cold saline and measured for wet weight. Three fixed lobes were fixed in 10% neutral buffered formalin for 24 hours. After fixation, these specimens proceeded to paraffin embedding for Masson's trichrome staining.

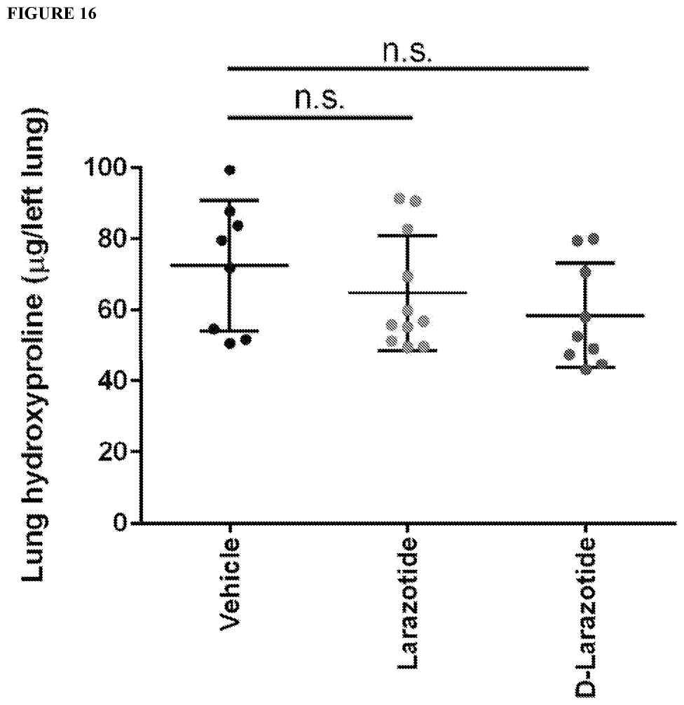

In order to quantify lung hydroxyproline content, frozen left lung samples were processed by an acid hydrolysis method as follows. Lung samples were acid-hydrolyzed with 300 μL of 6N HCl at 121° C. for 20 minutes, and neutralized with 300 μL of 4N NaOH containing 10 mg/mL activated carbon. AC buffer (2.2M acetic acid/0.48M citric acid, 300 μL) was then added to the samples, followed by centrifugation to collect the supernatant. A standard curve of hydroxyproline was constructed with 16, 8, 4, 2, 1 and 0.5 μg/mL of trans-4-hydroxy-L-proline (Sigma-Aldrich Co. LLC., USA Code: 54409). The prepared samples and standards (each 400 μL) were mixed with 400 μL chloramine T solution (NACALAI TESQUE, INC., Japan, Code: 08005-52) and incubated for 25 minutes at room temperature. The samples were then mixed with Ehrlich's solution (400 μL) and heated at 65° C. for 20 minutes to develop the color. After the samples were cooled on ice and centrifuged to remove precipitates, the optical density of each supernatant was measured at 560 nm. The concentrations of hydroxyproline were calculated from the hydroxyproline standard curve, as depicted in FIG. 16. Lung hydroxyproline contents are expressed as μg per left lung.

Histological analyses of the samples were also conducted. Right lung tissues prefixed in 10% neutral buffered formalin were embedded in paraffin and sectioned at 4 μm. For Masson's Trichrome staining, the sections were deparaffinized and rehydrated, followed by re-fixation with Bouin' s solution for 15 minutes. The sections were stained in Weigert's iron Hematoxylin working solution (Sigma-Aldrich), Biebrich scarlet-Acid fuchsin solution (SigmaAldrich), Phosphotungstic/phosphomolybdic Acid solution, Aniline blue solution, and 1% Acetic Acid solution (Sigma-Aldrich).

For quantitative analysis of lung fibrosis area, bright field images of Masson's Trichrome-stained sections were randomly captured using a digital camera (DFC295; Leica, Germany) at 100-fold magnification, and the subpleural regions in 20 fields/mouse were evaluated according to the criteria for grading lung fibrosis (Ashcroft, T., et al., J Clin Pathol, 1988; 41:467-70), as shown below:

TABLE 3
Criteria for grading lung fibrosis

| Grade of fibrosis | Histological features |
|---|---|
| 0 | Normal lung |
| 1 | Minimal fibrosis thickening of alveolar or bronchiolar |
| | walls |
| 2 | Moderate thickening of walls without obvious damage |
| 3 | to lung architecture |
| 4 | Increased fibrosis with definite damage to lung |
| 5 | structure and formation of fibrous bands or small |
| | fibrous masses |
| 6 | Severe distortion of structure and large fibrous areas; |
| 7 | “honeycomb lung” is placed in this category. |
| 8 | Total fibrous obliteration of the field. |

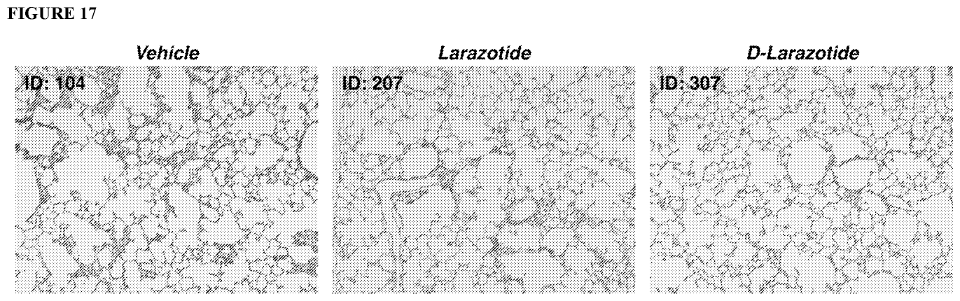

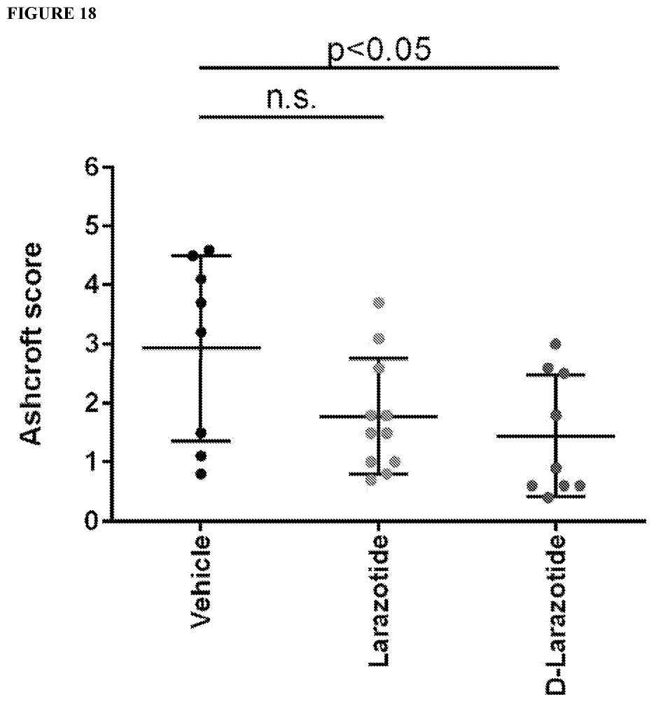

FIG. 17 shows sections of right lung tissue of mice that were administered vehicle, larazotide, and all d-larazotide, respectively, after being subjected to the staining process described above. FIG. 18 depicts the grading results of the stained right lung sections per the Ashcroft evaluation and grading criteria. The all d-larazotide right lung samples exhibit less fibrosis than either the vehicle or larazotide samples.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

What is claimed is:

1. A pharmaceutical composition comprising an effective amount of a peptide having the amino acid sequence Gly-Gly-Val-Leu-Val-Gln-Pro-Gly (SEQ ID NO: 1) with one or more (d)-amino acids to promote tight junction integrity, or a pharmaceutically acceptable salt thereof, and a pharmaceutically-acceptable carrier.

2. The pharmaceutical composition of claim 1, wherein the peptide has the amino acid sequence Gly-Gly-(d)Val-(d)Leu-(d)Val-(d)Gln-(d)Pro-Gly (SEQ ID NO: 6).

3. The pharmaceutical composition of claim 1, wherein the peptide has the amino acid sequence Gly-Gly-Val-Leu-Val-Gln-(d)Pro-Gly (SEQ ID NO: 9).

4. The pharmaceutical composition of any one of claims 1 to 3, wherein the composition contains less than about 0.5 mg of the peptide.

5. The pharmaceutical composition of claim 4, wherein the composition contains about 0.25 mg of the peptide or less.

6. The pharmaceutical composition of claim 4, wherein the composition contains from about 50 μg to about 400 μg of the peptide.

7. The Pharmaceutical composition of claim 4, wherein the composition contains from about 50 μg to about 150 μg of the peptide.

8. The pharmaceutical composition of any one of claims 1 to 3, wherein the composition contains more than about 0.5 mg of peptide.

9. The composition of claim 8, wherein the composition contains at least about 1.0 mg of peptide.

10. The composition of claim 8, wherein the composition contains at least about 2.0 mg of the peptide.

11. The pharmaceutical composition of any one of the previous claims, wherein the peptide is an acetate salt.

12. The pharmaceutical composition of any one of claims 1 to 11, wherein the composition is formulated for gastrointestinal delivery.

13. The pharmaceutical composition of claim 12, wherein the composition is formulated with an enteric coating for delivery of the peptide to the small intestine or large intestine.

14. The pharmaceutical composition of claim 13, wherein the composition is formulated for delivery of the peptide to one or more of the duodenum, jejunum, and/or the ileum.

15. The pharmaceutical composition of any one of claims 12 to 14, wherein the composition is formulated for delivery of the peptide to the large intestine.

16. The pharmaceutical composition of claim 15, wherein the composition is formulated for delivery of the peptide to one or more of the cecum, the ascending colon, the transverse colon, the descending colon, and/or the sigmoid colon.

17. The composition of any one of claims 12 to 16, where the composition contains peptide-containing beads with an enteric coating that is stable in gastric fluid and unstable in intestinal fluid so as to substantially release the peptide in the small intestine and/or large intestine.

18. The pharmaceutical composition of claim 17, wherein the pharmaceutical composition begins to release the peptide starting within about 15, about 30, or about 45 minutes, or about 60 minutes of exposure to simulated intestinal fluid.

19. The pharmaceutical composition of any one of claims 12 to 18, wherein the composition provides for a sustained release of the peptide in simulated intestinal fluid for at least about 180 minutes, or at least about 210 minutes, or at least about 240 minutes.

20. The pharmaceutical composition of claim 19, wherein the peptide is contained in a biodegradable or erodible matrix.

21. The pharmaceutical composition of claim 20, wherein the polymer matrix comprises a polysaccharide matrix.

22. The pharmaceutical composition of claim 21, wherein the matrix comprises one or more of cellulose, chitin, chitosan, alginate, amylose, pectin, callose, laminarin, chrysolaminarin, xylan, arabinoxylan, mannan, fucoidan, galactomannan, xanthan gum, dextran, welan gum, gellan gum, diutan gum, pullulan, hyaluronic acid, and derivatives thereof.

23. The composition of claim 21, wherein the matrix comprises microcrystalline cellulose.

24. The pharmaceutical composition of any one of claims 1 to 23, wherein the composition comprises one or more population of beads having a pH-dependent, delayed-release coating, which is optionally a 1:1 co-polymer of acrylate and methacrylate.

25. The pharmaceutical composition of any one of claims 19 to 23, wherein the composition comprises an enteric coating comprising a co-polymer of methyl acrylate, methyl methacrylate, and methacrylic acid.

26. The pharmaceutical composition of claim 25, wherein the ratio of free carbonyl groups to ester groups in the co-polymer is about 1:10.

27. The pharmaceutical composition of any one of claims 1 to 11, wherein the composition is formulated for parenteral administration.

28. The pharmaceutical composition of claim 27, wherein the composition is formulated for subcutaneous, intramuscular, or intravenous administration.

29. The pharmaceutical composition of any one of claims 1 to 11, wherein the composition is formulated for pulmonary administration.

30. The pharmaceutical composition of claim 29, wherein the composition is formulated as a solution aerosol or powder aerosol.

31. The pharmaceutical composition of any one of claims 1 to 11, wherein the composition is formulated for nasal administration.

32. The pharmaceutical composition of any one of claims 1 to 11, wherein the composition is formulated for ophthalmic administration.

33. A method for promoting tight junction integrity of a tissue, comprising administering a composition of any one of claims 1 to 32 to the tissue of a subject in need.

34. The method of claim 33, wherein the subject has or is at risk of a condition associated with epithelial permeability.

35. The method of claim 33 or 34, wherein the composition is administered once or twice daily, or once or twice weekly.

36. A method for treating or preventing a condition associated with gastrointestinal epithelial permeability in a subject, comprising, administering the composition of any one of claims 1 to 26 to the GI of said subject.

37. The method of claim 36, wherein the subject has celiac disease.

38. The method of claim 36, wherein the subject has Inflammatory Bowel Disease.

39. The method of claim 36, wherein the subject has ulcerative colitis.

40. The method of claim 36, wherein the subject has Crohn's disease.

41. The method of claim 36, wherein the subject has environmental enteropathy or necrotizing enterocolitis.

42. The method of claim 36, wherein the subject has intestinal ischemia.

43. The method of claim 36, wherein the subject has or is at risk of inflammatory liver disease.

44. The method of claim 43, wherein the subject has a fatty liver disease.

45. The method of claim 43, wherein the subject has non-alcoholic fatty liver disease (NAFLD) or non-alcoholic steatohepatitis (NASH).

46. The method of claim 36, wherein the subject has one or more of kidney disease, IgA nephropathy, viral hepatitis, diabetes, hypertriglyceridemia, and insulin resistance.

47. The method of claim 46, wherein the subject has diabetes mellitus.

48. The method of claim 36, wherein the subject has a pulmonary condition, which is optionally, asthma, chronic obstructive pulmonary disease (COPD), cystic fibrosis, lung cancer, and respiratory infection.

49. The method of claim 48, wherein the subject has or is at risk of pulmonary fibrosis.

50. The method of claim 49, wherein the pulmonary fibrosis is idiopathic pulmonary fibrosis or chemotherapy-induced lung fibrosis.

51. The method of claim 48, wherein the subject has an interstitial lung disease.

52. The method of claim 48, wherein the subject has or is at risk of Acute Lung Injury or Acute Respiratory Distress Syndrome.

53. The method of claim 52, wherein the subject has a coronavirus infection, which is optionally SARS-CoV2.

54. The method of any one of claims 36 to 53, wherein the composition is administered about once daily about twice daily, or about 3 times daily.

55. A method for treating a subject having or at risk of a pulmonary disease comprising administering the composition of any one of claims 29 to 31 to the patient.

56. The method of claim 55, wherein the patient has a pulmonary condition, which is optionally, asthma, chronic obstructive pulmonary disease (COPD), cystic fibrosis, lung cancer, and respiratory infection.

57. The method of claim 56, wherein the patient has or is at risk of pulmonary fibrosis.

58. The method of claim 57, wherein the pulmonary fibrosis is idiopathic pulmonary fibrosis or chemotherapy-induced lung fibrosis.

59. The method of claim 55, wherein the patient has an interstitial lung disease.

60. The method of claim 55, wherein the patient has or is at risk of Acute Lung Injury or Acute Respiratory Distress Syndrome.

61. The method of claim 60, wherein the patient has a coronavirus infection, which is optionally SARS-CoV2.

62. The method of claim 55, wherein the pulmonary disease is selected from asthma, chronic obstructive pulmonary disease (COPD), cystic fibrosis (CF), lung cancer, and respiratory infection.

63. The method of any one of claims 55 to 62, wherein the composition is administered about once daily about twice daily, or about 3 times daily.

64. A method for treating a subject having or at risk of Kawasaki disease, multisystem inflammatory syndrome in children (Mis-c), or systemic inflammatory response syndrome, comprising administering the composition of any one of claims 1 to 32 to the subject.

65. A method for treating a subject having or at risk of an autoimmune disease, optionally selected from rheumatoid arthritis, juvenile rheumatoid arthritis, lupus, type 1 diabetes, and multiple sclerosis, comprising administering the composition of any one of claims 1 to 32 to the subject.

66. A method for treating a subject having or at risk of a neoplasm, comprising administering the composition of any one of claims 1 to 33 to the subject.

67. The method of claim 66, wherein the subject further receives cancer immunotherapy.

68. The method of claim 67, wherein the cancer immunotherapy is therapy with an immune checkpoint inhibitor.

69. The method of claim 66, wherein the subject is receiving a chemotherapy.

70. The method of claim 69, wherein the subject has or is at risk of chemotherapy-induced colitis.

71. A method for treating a subject having or at risk of a condition of the central nervous system, comprising administering the composition of any one of claims 1 to 33 to the subject.

72. The method of claim 71, wherein the condition is Parkinson's disease, Alzheimer's disease, Multiple Sclerosis, and dementia.

73. A method for treating a subject having or at risk of a total parenteral nutrition (TPN)-induced disorder, comprising administering the composition of any one of claims 1 to 33 to the subject.

74. The method of claim 73, wherein the total parenteral nutrition (TPN)-induced disorder is selected from TPN-induced intestinal mucosal atrophy and TPN-induced liver disease.

75. The method of claim 73 or 74, wherein the composition is administered about once daily, about twice daily, or about three times daily.


SEQUENCE LISTING (9 sequences)

SEQ ID NO: 1 - protein, 7 aa, Artificial Sequence
  Synthetic polymer.
GVLVQPG

SEQ ID NO: 2 - protein, 8 aa, Artificial Sequence
  Synthetic polymer.
  (d)-amino acid (4)..(4)
GGVLVQPG

SEQ ID NO: 3 - protein, 9 aa, Artificial Sequence
  Synthetic polymer.
  (d)-amino acid (1)..(1)
VGGVLVQPG

SEQ ID NO: 4 - protein, 9 aa, Artificial Sequence
  Synthetic polymer.
VGGVLVQPG

SEQ ID NO: 5 - protein, 8 aa, Artificial Sequence
  Synthetic polymer.
  (d)-amino acid (3)..(3)
GGVLVQPG

SEQ ID NO: 6 - protein, 8 aa, Artificial Sequence
  Synthetic polymer.
  (d)-amino acid (3)..(3)
  (d)-amino acid (4)..(4)
  (d)-amino acid (5)..(5)
  (d)-amino acid (6)..(6)
  (d)-amino acid (7)..(7)
GGVLVQPG

SEQ ID NO: 7 - protein, 7 aa, Artificial Sequence
  Synthetic polymer.
GVLVQPG

SEQ ID NO: 8 - protein, 6 aa, Artificial Sequence
  Synthetic polymer.
VLVQPG

SEQ ID NO: 9 - protein, 8 aa, Artificial Sequence
  Synthetic polymer.
  (d)-amino acid (7)..(7)
GGVLVQPG